(19) United States
(12) Patent Application Publication
(10) Pub. No.: US 2022/0265212 A1
(43) Pub. Date: Aug. 25, 2022

(54) MEASURING SPATIAL WORKING MEMORY USING MOBILE-OPTIMIZED SOFTWARE TOOLS

(71) Applicant: Hoffmann-La Roche Inc., Little Falls, NJ (US)

(72) Inventors: Michael LINDEMANN, Basel (CH); Florian LIPSMEIER, Basel (CH); David NOBBS, Basel (CH); Wei-Yi CHENG, New York, NY (US); David Alexander SLATER, Basel (CH); Timothy KILCHENMANN, Basel (CH); Christian GOSSENS, Basel (CH); Joerg HIPP, Lorrach (DE); Christopher CHATHAM, San Francisco, CA (US); Eduardo Alberto APONTE PEREZ, Zurich (CH)

(73) Assignee: Hoffmann-La Roche Inc., Little Falls, NJ (US)

(21) Appl. No.: 17/685,084

(22) Filed: Mar. 2, 2022

Related U.S. Application Data
(63) Continuation of application No. PCT/US2020/049455, filed on Sep. 4, 2020.
(60) Provisional application No. 62/896,402, filed on Sep. 5, 2019.

(51) Int. Cl. A61B 5/00; G09B 19/00; G16H 10/20

(52) U.S. Cl. CPC A61B 5/4848; A61B 5/4088; G09B 19/00; G16H 10/20

(57) ABSTRACT

Aspects of the disclosure relate to mobile-optimized software tools that may be deployed and used to measure spatial working memory across diverse clinical trial populations. For example, some aspects describe computational optimization and participatory design of a novel spatial working memory task for clinical trials relating to autism spectrum disorders (ASD) or other neurological conditions (e.g., Alzheimer's disease). Software tools as described herein may be used for measuring treatment effects on spatial working memory and/or provide treatments to improve a patient's spatial working memory. Digital biomarkers may be generated for each patient based on the patient's spatial working memory.

### CROSS-REFERENCE TO RELATED APPLICATIONS

This application is a continuation of International Application No. PCT/US2020/049455, filed Sep. 4, 2020, which claims priority to U.S. Provisional Application No. 62/896,402, filed Sep. 5, 2019, the disclosures of which are hereby incorporated by reference in their entireties.

### TECHNICAL FIELD

Aspects described herein relate to digital health tools for medical diagnostics and analytics. In particular, one or more aspects described herein relate to mobile-optimized software tools that may be deployed and used to measure spatial working memory.

### BACKGROUND

Autism spectrum disorders (ASD) include a variety of neurodevelopmental conditions, including autism and Asperger syndrome. Individuals with ASD may have trouble with social communication and interaction and/or exhibit restricted and/or repetitive patterns of behavior. Those in the mild range of ASD may function independently, while those in the moderate to severe range may require substantial support in their daily lives. Those with ASD may experience deficits in social communication, have repetitive and limited interests, and/or exhibit sensory sensitivities. A central executive function tightly related with cognitive performance of patients with ASD is spatial working memory, i.e., the ability to hold and transform spatial information on demand. However, known methods of measuring spatial working memory require special equipment and/or specially trained personnel to perform each assessment, thereby raising costs for assessment and treatment.

### SUMMARY

The following presents a simplified summary of various aspects described herein. This summary is not an extensive overview, and is not intended to identify key or critical elements or to delineate the scope of the claims. The following summary merely presents some concepts in a simplified form as an introductory prelude to the more detailed description provided below. Corresponding apparatus, systems, and computer-readable media are also within the scope of the disclosure.

As a general introduction to the subject matter described herein, aspects of the disclosure relate to mobile-optimized software tools that may be deployed and used to measure spatial working memory across diverse clinical trial populations. For example, some aspects describe computational optimization and participatory design of a novel spatial working memory task for clinical trials in ASD. The administration of the autism-relevant neuropeptide vasopressin (and/or other treatments) may increase players' spatial working memory. Software tools as described herein may be used for measuring treatment effects on spatial working memory and/or provide treatments to improve a patient's spatial working memory. Digital biomarkers may be generated for each patient based on the patient's spatial working memory.

In one aspect of the disclosure, a computer-implemented method includes obtaining, from a plurality of mobile devices associated with a plurality of patients in a patient population, a plurality of game results, wherein each game result in the plurality of game results including game boards, moves provided by a patient associated with the mobile device providing the game result, and a difficulty for each of the game boards and the patient population includes a treatment administered to each patient in the patient population and at least one characteristic in common between each patient in the patient population, determining, based on the plurality of game results, the moves provided by the patient for each of the plurality of game results, and the difficulty for each of the game boards in the game results, sorted game results, iteratively generating representative parameters for each of the plurality of game results, and determining, for each patient and based on the iteratively generated representative parameters associated with the patient, a spatial working memory score for the patient.

In yet another aspect of the disclosure, the computer-implemented method further includes obtaining a historical memory score for each patient in the patient population and determining, based on a comparison of the historical memory score and the spatial working memory score for each patient in the patient population, a treatment effectiveness for the administered treatment.

In still another aspect of the disclosure, the administered treatment includes a therapeutically effective dose of a drug selected from the group of Arbaclofen, Balovaptan, a GABA-Aa5 PAM, a GABA-A1 modulator, a mGlu4/7 PAM, a Dopamine 2 receptor antagonist, in particular Risperidone, mu-opioid receptor antagonist, in particular naloxone, and/or NMDA glutamate receptor antagonist, in particular memantine, and pharmaceutically acceptable salts thereof.

In yet still another aspect of the disclosure, the computer-implemented method further includes obtaining, from a second plurality of mobile devices associated with a second plurality of patients in a second patient population, a second plurality of game results, wherein each game result in the second plurality of game results including game boards and moves provided by a patient associated with the mobile device providing the game result, the second patient population includes a second treatment administered to each patient in the second plurality of patients and at least one second characteristic in common between each patient in the second patient population, determining, for each patient in the second patient population, a second spatial working memory score for each patient in the second patient population, and determining the treatment effectiveness for the administered treatment is further based on the second spatial working memory score for each patient in the second patient population.

In yet another additional aspect of the disclosure, the second treatment includes a placebo and the at least one second characteristic of the second patient population is the same as at least one characteristic of the patient population.

In still another additional aspect of the disclosure, the representative parameters are iteratively generated using a procedure selected from the group of Markov Chain Monte Carlo simulations, grid search, and Bayesian estimation.

In yet still another additional aspect of the disclosure, the computer-implemented method further includes determining, based on the treatment effectiveness, a treatment recommendation for patients having a characteristic in common the with at least one characteristic of the patient population.

Yet another aspect of the disclosure includes a computing device including a processor and a memory in communication with the processor and storing instructions that, when read by the processor, cause the computing device to obtain, from a plurality of mobile devices associated with a plurality of patients in a patient population, a plurality of game results, wherein each game result in the plurality of game results including game boards, moves provided by a patient associated with the mobile device providing the game result, and a difficulty for each of the game boards and the patient population includes a treatment administered to each patient in the patient population and at least one characteristic in common between each patient in the patient population, determine, based on the plurality of game results, the moves provided by the patient for each of the plurality of game results, and the difficulty for each of the game boards in the game results, sorted game results, iteratively generate representative parameters for each of the plurality of game results, and determine, for each patient and based on the iteratively generated representative parameters associated with the patient, a spatial working memory score for the patient.

In yet another aspect of the disclosure, the instructions, when read by the processor, further cause the computing device to obtain a historical memory score for each patient in the patient population and determine, based on a comparison of the historical memory score and the spatial working memory score for each patient in the patient population, a treatment effectiveness for the administered treatment.

In still another aspect of the disclosure, the administered treatment includes a therapeutically effective dose of a drug selected from the group of Arbaclofen, Balovaptan, a GABA-Aa5 PAM, a GABA-A1 modulator, a mGlu4/7 PAM, a Dopamine 2 receptor antagonist, in particular Risperidone, mu-opioid receptor antagonist, in particular naloxone, and/or NMDA glutamate receptor antagonist, in particular memantine, and pharmaceutically acceptable salts thereof.

In yet still another aspect of the disclosure, the instructions, when read by the processor, further cause the computing device to obtain, from a second plurality of mobile devices associated with a second plurality of patients in a second patient population, a second plurality of game results, wherein each game result in the second plurality of game results including game boards and moves provided by a patient associated with the mobile device providing the game result, the second patient population includes a second treatment administered to each patient in the second plurality of patients and at least one second characteristic in common between each patient in the second patient population, determine, for each patient in the second patient population, a second spatial working memory score for each patient in the second patient population, and determine the treatment effectiveness for the administered treatment is further based on the second spatial working memory score for each patient in the second patient population.

In yet another additional aspect of the disclosure, the second treatment includes a placebo and the at least one second characteristic of the second patient population is the same as at least one characteristic of the patient population.

In still another additional aspect of the disclosure, the representative parameters are iteratively generated using a procedure selected from the group of Markov Chain Monte Carlo simulations, grid search, and Bayesian estimation.

In yet still another additional aspect of the disclosure, the instructions, when read by the processor, further cause the computing device to determine, based on the treatment effectiveness, a treatment recommendation for patients having a characteristic in common the with at least one characteristic of the patient population.

Still another aspect of the disclosure includes a non-transitory machine-readable medium storing instructions that, when executed by one or more processors, cause the one or more processors to perform steps including obtaining, from a plurality of mobile devices associated with a plurality of patients in a patient population, a plurality of game results, wherein each game result in the plurality of game results including game boards, moves provided by a patient associated with the mobile device providing the game result, and a difficulty for each of the game boards and the patient population includes a treatment administered to each patient in the patient population and at least one characteristic in common between each patient in the patient population, determining, based on the plurality of game results, the moves provided by the patient for each of the plurality of game results, and the difficulty for each of the game boards in the game results, sorted game results, iteratively generating representative parameters for each of the plurality of game results, and determining, for each patient and based on the iteratively generated representative parameters associated with the patient, a spatial working memory score for the patient.

In yet another aspect of the disclosure, the instructions, when executed by one or more processors, further cause the one or more processors to perform steps including obtaining a historical memory score for each patient in the patient population and determining, based on a comparison of the historical memory score and the spatial working memory score for each patient in the patient population, a treatment effectiveness for the administered treatment.

In still another aspect of the disclosure, the administered treatment includes a therapeutically effective dose of a drug selected from the group of Arbaclofen, Balovaptan, a GABA-Aa5 PAM, a GABA-A1 modulator, a mGlu4/7 PAM, a Dopamine 2 receptor antagonist, in particular Risperidone, mu-opioid receptor antagonist, in particular naloxone, and/or NMDA glutamate receptor antagonist, in particular memantine, and pharmaceutically acceptable salts thereof.

In yet still another aspect of the disclosure, the instructions, when executed by one or more processors, further cause the one or more processors to perform steps including obtaining, from a second plurality of mobile devices associated with a second plurality of patients in a second patient population, a second plurality of game results, wherein each game result in the second plurality of game results including game boards and moves provided by a patient associated with the mobile device providing the game result, the second patient population includes a second treatment administered to each patient in the second plurality of patients and at least one second characteristic in common between each patient in the second patient population, determining, for each patient in the second patient population, a second spatial working memory score for each patient in the second patient population, and determining the treatment effectiveness for the administered treatment is further based on the second spatial working memory score for each patient in the second patient population.

In yet another additional aspect of the disclosure, the second treatment includes a placebo and the at least one second characteristic of the second patient population is the same as at least one characteristic of the patient population.

In still another additional aspect of the disclosure, the instructions, when executed by one or more processors, further cause the one or more processors to perform steps including determining, based on the treatment effectiveness, a treatment recommendation for patients having a characteristic in common the with at least one characteristic of the patient population.

Yet another aspect of the disclosure includes a computer-implemented method for playing an interactive game including displaying a game board for a current round of the interactive game, the game board including a plurality of interactive elements and a goal location and the number of interactive elements is determined based on a spatial working memory score for a user, determining a first item location in the game board, wherein the first item location corresponds to a first interactive element of the plurality of interactive elements, obtaining a first user move, wherein the first user move identifies an interactive element in the plurality of interactive elements, determining that the interactive element identified in the first user move corresponds to the first item location, updating the interactive element identified in the first user move to indicate that the interactive element has been associated with an item location, determining a second item location in the game board, wherein the second item location corresponds to a second interactive element of the plurality of interactive elements distinct from the first interactive element, obtaining a second user move, wherein the user move identifies an interactive element in the plurality of interactive elements, determining that the interactive element identified in the second user move corresponds to the second item location, updating the interactive element identified in the second user move to indicate that the second interactive element has been associated with an item location, determining the current round of the interactive game is over based on each interactive element in the plurality of interactive elements having been associated with an item location, transmitting the obtained user moves and the game board to a remote server system, and displaying a game summary for the current round of the interactive game.

In yet another aspect of the disclosure, the computer-implemented method further includes obtaining, based on the determining that the interactive element identified in the first user move corresponds to the first item location, a third user move associating the first item location with the goal location.

In still another aspect of the disclosure, the computer-implemented method further includes generating a second game board based on the spatial working memory score for the user, obtaining user input indicating the user wishes to proceed to a next round of the interactive game, and displaying the second game board.

In yet still another aspect of the disclosure, the game board is further generated based on characteristics of a mobile device providing the interactive game and the characteristics of the mobile device are selected from the group of a screen size, a screen resolution, a pixel density, and available input devices.

In yet another additional aspect of the disclosure, the computer-implemented method further includes determining, based on the game board, the first user move, and the second user move, an updated spatial working memory score for the user, measuring, based on the spatial working memory score and the updated spatial working memory score, a treatment effect for the user, determining, based on the treatment effect, a therapeutically effective dosage of a drug for the user, and administering the therapeutically effective dosage of the drug to the user.

In still another additional aspect of the disclosure, the drug is selected from the group of Arbaclofen, Balovaptan, a GABA-Aa5 PAM, a GABA-A1 modulator, a mGlu4/7 PAM, a Dopamine 2 receptor antagonist, in particular Risperidone, mu-opioid receptor antagonist, in particular naloxone, and/or NMDA glutamate receptor antagonist, in particular memantine, and pharmaceutically acceptable salts thereof.

In yet still another additional aspect of the disclosure, the spatial working memory score for the user is determined based on a number of within-search errors and a number of between-search errors for a historical game session played by the user.

Still another aspect of the disclosure includes a computing device including a processor, and a memory in communication with the processor and storing instructions that, when read by the processor, cause the computing device to display a game board for a current round of an interactive game, the game board including a plurality of interactive elements and a goal location and the number of interactive elements is determined based on a spatial working memory score for a user, determine a first item location in the game board, wherein the first item location corresponds to a first interactive element of the plurality of interactive elements, obtain a first user move, wherein the first user move identifies an interactive element in the plurality of interactive elements, determine that the interactive element identified in the first user move corresponds to the first item location, update the interactive element identified in the first user move to indicate that the interactive element has been associated with an item location, determine a second item location in the game board, wherein the second item location corresponds to a second interactive element of the plurality of interactive elements distinct from the first interactive element, obtain a second user move, wherein the user move identifies an interactive element in the plurality of interactive elements, determine that the interactive element identified in the second user move corresponds to the second item location, update the interactive element identified in the second user move to indicate that the second interactive element has been associated with an item location, determine the current round of the interactive game is over based on each interactive element in the plurality of interactive elements having been associated with an item location, transmit the obtained user moves and the game board to a remote server system, and display a game summary for the current round of the interactive game.

In yet another aspect of the disclosure, the instructions, when read by the processor, further cause the computing device to obtain, based on the determining that the interactive element identified in the first user move corresponds to the first item location, a third user move associating the first item location with the goal location.

In still another additional aspect of the disclosure, the instructions, when read by the processor, further cause the computing device to generate a second game board based on the spatial working memory score for the user, obtain user input indicating the user wishes to proceed to a next round of the interactive game, and display the second game board.

In yet still another additional aspect of the disclosure, the game board is further generated based on characteristics of a mobile device providing the interactive game and the characteristics of the mobile device are selected from the group of a screen size, a screen resolution, a pixel density, and available input devices.

In yet another additional aspect of the disclosure, the instructions, when read by the processor, further cause the computing device to determine, based on the game board, the first user move, and the second user move, an updated spatial working memory score for the user, measure, based on the spatial working memory score and the updated spatial working memory score, a treatment effect for the user, determine, based on the treatment effect, a therapeutically effective dosage of a drug for the user, and administer the therapeutically effective dosage of the drug to the user.

In still another additional aspect of the disclosure, the drug is selected from the group of Arbaclofen, Balovaptan, a GABA-Aa5 PAM, a GABA-A1 modulator, a mGlu4/7 PAM, a Dopamine 2 receptor antagonist, in particular Risperidone, mu-opioid receptor antagonist, in particular naloxone, and/or NMDA glutamate receptor antagonist, in particular memantine, and pharmaceutically acceptable salts thereof.

In yet still another additional aspect of the disclosure, the spatial working memory score for the user is determined based on a number of within-search errors and a number of between-search errors for a historical game session played by the user.

Yet another aspect of the disclosure includes a non-transitory machine-readable medium storing instructions that, when executed by one or more processors, cause the one or more processors to perform steps including displaying a game board for a current round of an interactive game, the game board including a plurality of interactive elements and a goal location and the number of interactive elements is determined based on a spatial working memory score for a user, determining a first item location in the game board, wherein the first item location corresponds to a first interactive element of the plurality of interactive elements, obtaining a first user move, wherein the first user move identifies an interactive element in the plurality of interactive elements, determining that the interactive element identified in the first user move corresponds to the first item location, updating the interactive element identified in the first user move to indicate that the interactive element has been associated with an item location, determining a second item location in the game board, wherein the second item location corresponds to a second interactive element of the plurality of interactive elements distinct from the first interactive element, obtaining a second user move, wherein the user move identifies an interactive element in the plurality of interactive elements, determining that the interactive element identified in the second user move corresponds to the second item location, updating the interactive element identified in the second user move to indicate that the second interactive element has been associated with an item location, determining the current round of the interactive game is over based on each interactive element in the plurality of interactive elements having been associated with an item location, transmitting the obtained user moves and the game board to a remote server system, and displaying a game summary for the current round of the interactive game.

In yet another aspect of the disclosure, the instructions, when executed by one or more processors, further cause the one or more processors to perform steps including obtaining, based on the determining that the interactive element identified in the first user move corresponds to the first item location, a third user move associating the first item location with the goal location.

In still another aspect of the disclosure, the instructions, when executed by one or more processors, further cause the one or more processors to perform steps including generating a second game board based on the spatial working memory score for the user, obtaining user input indicating the user wishes to proceed to the next round of the interactive game, and displaying the second game board.

In yet still another additional aspect of the disclosure, the game board is further generated based on characteristics of a mobile device providing the interactive game and the characteristics of the mobile device are selected from the group of a screen size, a screen resolution, a pixel density, and available input devices.

In yet another additional aspect of the disclosure, the instructions, when executed by one or more processors, further cause the one or more processors to perform steps including determining, based on the game board, the first user move, and the second user move, an updated spatial working memory score for the user, measuring, based on the spatial working memory score and the updated spatial working memory score, a treatment effect for the user, determining, based on the treatment effect, a therapeutically effective dosage of a drug for the user, the drug selected from the group of Arbaclofen, Balovaptan, a GABA-Aa5 PAM, a GABA-A1 modulator, a mGlu4/7 PAM, a Dopamine 2 receptor antagonist, in particular Risperidone, mu-opioid receptor antagonist, in particular naloxone, and/or NMDA glutamate receptor antagonist, in particular memantine, and pharmaceutically acceptable salts thereof, and administering the therapeutically effective dosage of the drug to the user.

In still another additional aspect of the disclosure, the spatial working memory score for the user is determined based on a number of within-search errors and a number of between-search errors for a historical game session played by the user.

Still another aspect of the disclosure includes a computer-implemented method of generating a digital biomarker including determining a task difficulty level for assessment of a patient having a neurological condition, generating an interactive task at the task difficulty level, generating for display on a mobile device a graphical user interface for receiving task input from the patient attempting to complete the task, determining a task outcome based on the received task input, generating a modified task difficultly level based on the received task input, iterating through the previous generating and determining the task outcome steps using the modified task difficulty level until a predetermined condition is met, and based on the predetermined condition being met, determining a digital biomarker for the patient by analyzing the plurality of received task inputs and determined task outcomes.

In yet another aspect of the disclosure, the predetermined condition includes the patient completing the task at a predetermined difficulty level.

In still another aspect of the disclosure, the predetermined condition includes the patient making at least a predetermined number of errors while attempting to complete any task.

In yet still another aspect of the disclosure, the interactive task includes hiding an object on a game board, and wherein each difficulty is associated with a different number of interactive elements on the game board.

In yet another additional aspect of the disclosure, determining the digital biomarker is performed by a server device configured to receive task input and task outcomes.

In still another additional aspect of the disclosure, the patient is selected from a population that has been administered a treatment and the method further includes obtaining a historical digital biomarker for each patient in the in the population, generating a new digital biomarker, using the previously recited steps, for each patient in the population, and determining, based on a comparison of the historical digital biomarker and the new digital biomarker for each patient, a treatment effectiveness for the administered treatment.

In yet still another additional aspect of the disclosure, generating the modified task difficulty level includes using an iterative procedure selected from the group consisting of Markov Chain Monte Carlo simulations, grid search, and Bayesian estimation.

Yet another aspect of the disclosure includes a non-transitory machine-readable medium storing instructions that, when executed by one or more processors, cause the one or more processors to perform steps including determining a task difficulty level for assessment of a patient having a neurological condition, generating an interactive task at the task difficulty level, generating for display on a mobile device a graphical user interface for receiving task input from the patient attempting to complete the task, determining a task outcome based on the received task input, generating a modified task difficultly level based on the received task input, iterating through the previous generating and determining the task outcome steps using the modified task difficulty level until a predetermined condition is met, and based on the predetermined condition being met, determining a digital biomarker for the patient by analyzing the plurality of received task inputs and determined task outcomes.

In yet another aspect of the disclosure, the predetermined condition includes the patient completing the task at a predetermined difficulty level.

In still another aspect of the disclosure, the predetermined condition includes the patient making at least a predetermined number of errors while attempting to complete any task.

In yet still another aspect of the disclosure, the interactive task includes hiding an object on a game board, and wherein each difficulty is associated with a different number of interactive elements on the game board.

In yet another additional aspect of the disclosure, determining the digital biomarker is performed by a server device configured to receive task input and task outcomes.

In still another additional aspect of the disclosure, the instructions, when executed by one or more processors, further cause the one or more processors to perform steps including obtaining a historical digital biomarker for each patient in the in the population, wherein each patient is selected from a population that has been administered a treatment, generating a new digital biomarker, using the previously recited steps, for each patient in the population, and determining, based on a comparison of the historical digital biomarker and the new digital biomarker for each patient, a treatment effectiveness for the administered treatment.

In yet still another additional aspect of the disclosure, generating the modified task difficulty level includes using an iterative procedure selected from the group consisting of Markov Chain Monte Carlo simulations, grid search, and Bayesian estimation.

These features, along with many others, are discussed in greater detail below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more complete understanding of aspects described herein and the advantages thereof may be acquired by referring to the following description in consideration of the accompanying drawings, in which like reference numbers indicate like features, and wherein:

FIG. 1 depicts a custom computing system and architecture which may be used to implement one or more illustrative aspects described herein;

FIG. 2A depicts one or more design tradeoffs in developing assessments for ASD according to one or more illustrative aspects described herein;

FIG. 2B is a conceptual illustration of spatial working memory according to one or more illustrative aspects described herein;

FIGS. 3A-3P depict example graphical user interfaces that may be presented by a software tool that may be deployed and used to measure spatial working memory across diverse clinical trial populations according to one or more illustrative aspects described herein;

FIG. 4A is a flowchart conceptually illustrating a process for using a software tool according to one or more illustrative aspects described herein;

FIG. 4B is a flowchart conceptually illustrating a process for dynamically generating a user interface according to one or more illustrative aspects described herein;

FIG. 4C is a flowchart conceptually illustrating a process for deriving working memory scores given individual data according to one or more illustrative aspects described herein;

FIG. 4D is pseudocode conceptually illustrating a process for deriving working memory scores given individual data according to one or more illustrative aspects described herein;

FIG. 4E is a conceptual illustration of responses provided by a patient playing an interactive game according to one or more illustrative aspects described herein;

FIG. 5A is a flowchart conceptually illustrating a process for analyzing spatial working memory according to one or more illustrative aspects described herein

FIG. 5B is a flowchart conceptually illustrating a process for administering treatments according to one or more illustrative aspects described herein;

FIG. 6 depicts a set of challenges for autistic individuals that may be related to spatial working memory according to one or more illustrative aspects described herein;

FIG. 7 depicts example data associated with neuropsychological tests of spatial working memory according to one or more illustrative aspects described herein;

FIG. 8 and FIG. 9 depict focus group findings associated with a software tool that may be deployed and used to measure spatial working memory across diverse clinical trial populations according to one or more illustrative aspects described herein;

FIG. 10 and FIG. 11 depict model results associated with a software tool that may be deployed and used to measure spatial working memory across diverse clinical trial populations according to one or more illustrative aspects described herein; and

FIG. 12 depicts a neural model associated with a software tool that may be deployed and used to measure spatial working memory across diverse clinical trial populations according to one or more illustrative aspects described herein.

### DETAILED DESCRIPTION

In the following description of the various embodiments, reference is made to the accompanying drawings, which form a part hereof, and in which is shown by way of illustration various embodiments in which aspects described herein may be practiced. It is to be understood that other embodiments may be utilized and structural and functional modifications may be made without departing from the scope of the described aspects and embodiments. Aspects described herein are capable of other embodiments and of being practiced or being carried out in various ways. It is to be understood that the phraseology and terminology used herein are for the purpose of description and should not be regarded as limiting. Rather, the phrases and terms used herein are to be given their broadest interpretation and meaning. The use of “including” and “comprising” and variations thereof is meant to encompass the items listed thereafter and equivalents thereof as well as additional items and equivalents thereof. The use of the terms “mounted,” “connected,” “coupled,” “positioned,” “engaged” and similar terms, is meant to include both direct and indirect mounting, connecting, coupling, positioning, and engaging.

As a general introduction to the subject matter described herein, aspects of the disclosure relate to mobile-optimized software tools that may be deployed and used to measure spatial working memory across diverse clinical trial populations such as, but not limited to, autism The autism spectrum disorder (ASD) is defined by deficits in social communication, repetitive and limited interests, and/or sensory sensitivities. In addition, cognitive deficits are common and play an important role in ASD patients' well-being. For example, these deficits are predictive of impaired daily life functioning and employability and therefore have an impact on patients and their caregivers. Hence, in devising therapies to treat conditions of the central nervous system, it is paramount to monitor and evaluate the effects of such therapies on cognitive functioning. A central executive function tightly related with cognitive performance that may be monitored and evaluated without laboratory tests is spatial working memory, i.e., the ability to hold, update, and transform visual and spatial information on demand.

To evaluate visual and/or spatial working memory, existing techniques are mostly restricted to change detection tests (in which patient are asked to identified aspects of a static scene that change from one presentation to the other) to measure the precision in matching paradigms (in which patients have to reproduce a feature of a visual scene) and to evaluate the influence of distractors in memory encoding. These tests do not evaluate the dynamics of spatial working memory, i.e., how items are stored, retrieved and removed from memory as the complexity of visual scenes increase, is not well understood and there is no prior art suggesting how to measure the spatial working memory capacity when patients are required to store, delete and update items in memory.

Discrete slots models have been extraordinarily successful in explaining spatial working memory (SWM) in single trial task as the change detection task. This approach has been successfully extended to paradigms that query the precision of the items stored in visual memory. Less is known about the dynamics of SWM in situations that require continuous storage and update of items across several trials. The computational analysis described herein clearly demonstrates two general points: items are stored in two largely independent storages and retrieval of items is stochastic. A within-search priority model may generate very different error rate statistics without assuming independent storages. Moreover, the marginal error rate is not a linear function of difficulty. Slot models of SWM typically assume that that storage and retrieval are deterministic operations, but the contents stored are thought to be random. Aspects described herein conclusively rule out this hypothesis. In particular, the theoretical error rates predicted by such a model are larger than observed in real-world data generated using systems as described herein. The models proposed herein suggest that all items are stored in memory, but these may only be retrieved correctly from memory in fixed number of occasions. This shows that the failure rate from which items are retrieved from memory is relatively constant across set sizes.

In accordance with aspects of the disclosure, an interactive game specifically optimized for mobile devices is used to perform a variety of tasks. The interactive game is designed to be visually appealing and to improve the psychometric properties of the original task. The evaluation of the interactive game utilizes a novel computational approach based on the discrete slot model of spatial working memory that accurately captures the behavior of the users. Based on the user behavior, the effectiveness of administered treatments may be determined. Further, recommended treatments may be determined and/or administered to the user to improve the user's SWM. It should be also be noted that a variety of disorders and diseases, such as Alzheimer's, may also exhibit effects on the cognitive ability of patients. The processes and techniques described herein are described with respect to ASD, but it should be noted that the processes and techniques described may be used to evaluate spatial working memory and provide treatments to patients suffering from any of a variety of disorders and diseases affecting the patient's cognitive abilities and/or brain activity.

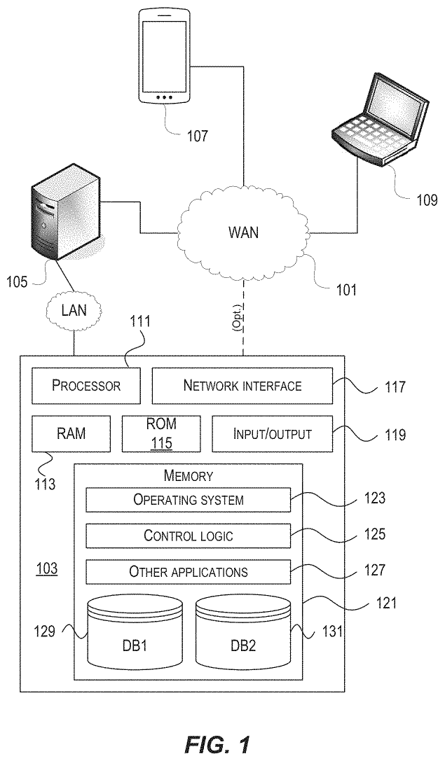

FIG. 1 illustrates one example of a custom network architecture and data processing devices that may be used to implement one or more illustrative aspects described herein. Various network nodes 103, 105, 107, and 109 may be interconnected via a wide area network (WAN) 101, such as the Internet. Other networks may also or alternatively be used, including private intranets, corporate networks, LANs, wireless networks, personal networks (PAN), and the like. Network 101 is for illustration purposes and may be replaced with fewer or additional computer networks. A local area network (LAN) may have one or more of any known LAN topology and may use one or more of a variety of different protocols, such as Ethernet. Devices 103, 105, 107, 109 and other devices (not shown) may be connected to one or more of the networks via twisted pair wires, coaxial cable, fiber optics, radio waves, or other communication media.

The term “network” as used herein and depicted in the drawings refers not only to systems in which remote storage devices are coupled together via one or more communication paths, but also to stand-alone devices that may be coupled, from time to time, to such systems that have storage capability. Consequently, the term “network” includes not only a “physical network” but also a “content network,” which includes the data—attributable to a single entity—which resides across all physical networks.

The components may include data server 103, web server 105, and client devices 107, 109. Data server 103 provides overall access, control, and administration of databases and control software for performing one or more illustrative aspects described herein. Data server 103 may be connected to web server 105 through which users interact with and obtain data as requested. Alternatively, data server 103 may act as a web server itself and be directly connected to the Internet (in which case device 105 is not needed). Data server 103 may be connected to web server 105 through the network 101 (e.g., the Internet), via direct or indirect connection, or via some other network. Users may interact with the data server 103 using remote computers 107, 109, e.g., using an application, mobile app, or web browser to connect to the data server 103 via one or more externally exposed web sites and/or web services hosted by web server 105. Client computers 107, 109 may be used in concert with data server 103 to access data stored therein, or may be used for other purposes. For example, from client device 107 a user may access web server 105 using an Internet browser or by executing a software application that communicates with web server 105 and/or data server 103 over a computer network (such as the Internet).

Servers and applications may be combined on the same physical machines, and retain separate virtual or logical addresses, or may reside on separate physical machines. FIG. 1 illustrates just one example of a network architecture that may be used, and those of skill in the art will appreciate that the specific network architecture and data processing devices used may vary, and are secondary to the functionality that they provide, as further described herein. For example, services provided by web server 105 and data server 103 may be combined on a single server.

Each component 103, 105, 107, 109 may be any type of computer, server, or data processing device. Data server 103, e.g., may include a processor 111 controlling overall operation of the data server 103. Data server 103 may further include RAM 113, ROM 115, network interface 117, input/output interfaces 119 (e.g., keyboard, mouse, display, printer, etc.), and memory 121. I/O 119 may include a variety of interface units and drives for reading, writing, displaying, and/or printing data or files. Memory 121 may further store operating system software 123 for controlling overall operation of the data processing device 103, control logic 125 for instructing data server 103 to perform aspects described herein, and other application software 127 providing secondary, support, and/or other functionality which may or may not be used in conjunction with other aspects described herein. The control logic may also be referred to herein as the data server software 125. Functionality of the data server software may refer to operations or decisions made automatically based on rules coded into the control logic, made manually by a user providing input into the system, and/or a combination of automatic processing based on user input (e.g., queries, data updates, etc.).

Memory 121 may also store data used in performance of one or more aspects described herein, including a first database 129 and a second database 131. In some embodiments, the first database may include the second database (e.g., as a separate table, report, etc.). That is, the information may be stored in a single database, or separated into different logical, virtual, or physical databases, depending on system design. Devices 105, 107, 109 may have similar or different architecture as described with respect to device 103. Those of skill in the art will appreciate that the functionality of data processing device 103 (or device 105, 107, 109) as described herein may be spread across multiple data processing devices, for example, to distribute processing load across multiple computers, to segregate transactions based on geographic location, user access level, quality of service (QoS), etc.

One or more aspects described herein may be embodied in computer-usable or readable data and/or computer-executable instructions, such as in one or more program modules, executed by one or more computers or other devices as described herein. Program modules may include routines, programs, objects, components, data structures, etc. that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer readable medium such as a hard disk, optical disk, removable storage media, solid state memory, RAM, etc. As will be appreciated by one of skill in the art, the functionality of the program modules may be combined or distributed as desired in various embodiments. In addition, the functionality may be embodied, in whole or in part, in firmware or hardware equivalents such as integrated circuits, field programmable gate arrays (FPGA), and the like. Particular data structures may be used to more effectively implement one or more aspects, and such data structures are contemplated within the scope of computer executable instructions and computer-usable data described herein.

Interactive games and/or other mobile-optimized software tools may be deployed and used to measure spatial working memory (SWM) across diverse clinical and non-clinical populations in accordance with one or more aspects of the disclosure. For instance, such tools may create a sensitive and robust measure of SWM for diverse clinical trial populations. Additionally or alternatively, such tools may enable attainment of a higher signal-to-noise ratio in measurement data via frequent and/or remote assessment on mobile devices. Additionally or alternatively, such tools may ensure acceptability by individuals for use in clinical trials. Additionally or alternatively, such tools may optimize detection power for individual differences and longitudinal effects that may arise in assessment processes.

In some instances, mobile-optimized software tools that may be deployed and used to measure spatial working memory across diverse clinical trial populations in accordance with one or more aspects of the disclosure may implement certain methods. For instance, such tools may provide a prototype based on multinational consortia and participatory focus groups. Additionally or alternatively, such tools may adapt the task parameters (which may be associated with assessments) for mobile devices and a wide range of abilities. Additionally or alternatively, such tools may optimize the task's sensitivity to SWM dynamics with Monte Carlo methods. Additionally or alternatively, such tools may validate the task in observational and interventional trials in clinical and non-clinical populations.

One or more aspects of the disclosure provide a novel test of spatial working memory that is optimized for mobile device-based remote deployment. In addition, one or more aspects of the disclosure provide a novel test of spatial working memory that is suitable for cognitively diverse individuals as young as age seven, as old as age ninety, or with intelligent quotients (IQ) below 70 and/or above 140. Further, one or more aspects of the disclosure provide a novel test of SWM that is efficient and sensitive in agent-based models as well as amenable to neurophysiological modeling of excitatory/inhibitory balance. One rationale underlying one or more aspects of the disclosure is to provide remote, frequent assessments in clinical trials. In particular, it is desirable to provide assessments that are sensitive to individual differences and interventions, appropriate for diverse cognitive ability, scalable for global deployment (e.g., via mobile devices), and/or well characterized through rigorous validation and formal modeling.

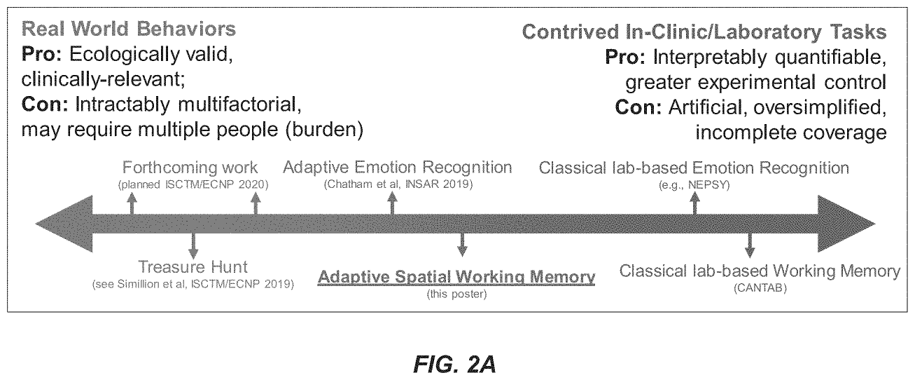

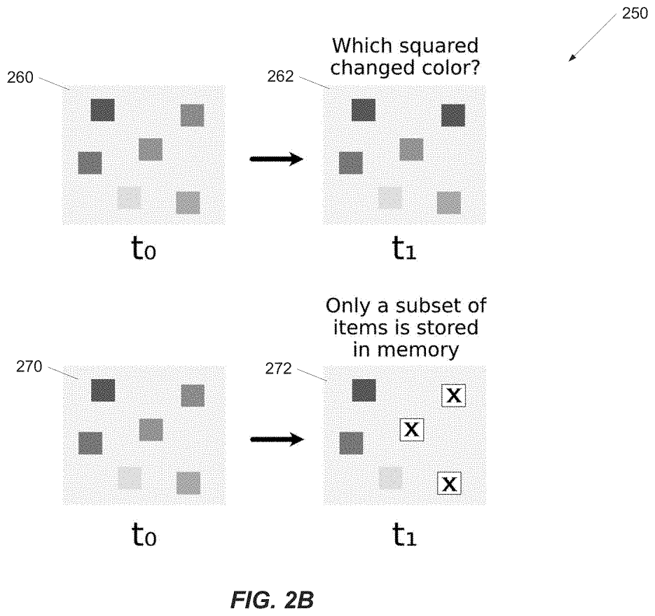

In designing such assessments for clinical and non-populations, a design tradeoff between ecologically valid manifestations of core symptoms and their tractability may be encountered. This design tradeoff is illustrated in FIG. 2A. Some aspects of the disclosure focus on an adaptive spatial working memory task that may be seen as lying close to the midpoint of the range. FIG. 2B is a conceptual illustration of spatial working memory (SWM) according to one or more illustrative aspects described herein. SWM has been suggested to include a finite set of discrete slots, in which new information is stored as long as enough slots are available. The conceptual illustration 250 includes a set of blocks 260 at time to. At time t1, one of the blocks changes color as shown in set of blocks 262. For example, the top right most block changed color from dark green at time t0 to maroon at time t1. The spatial working memory 270 at time t0 shows that the user sees all six blocks in set of blocks 260 and their colors. However, at time t1, spatial working memory 272 shows that the user may only recall the colors of the three leftmost blocks, and the three rightmost blocks (as indicated by an ‘x’) are of unknown color. That is, the SWM of the user is three blocks. As such, the user is unlikely to be able to accurately identify which of the blocks changed color between time t0 and time t1.

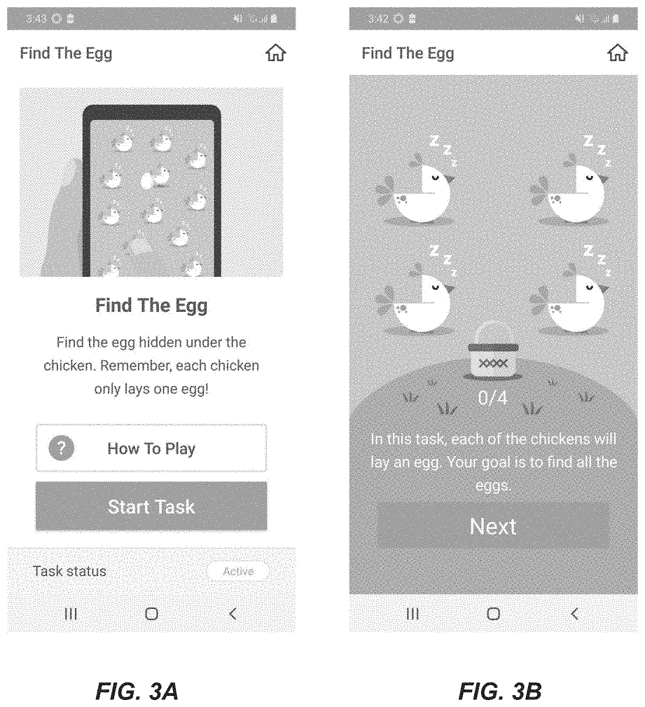

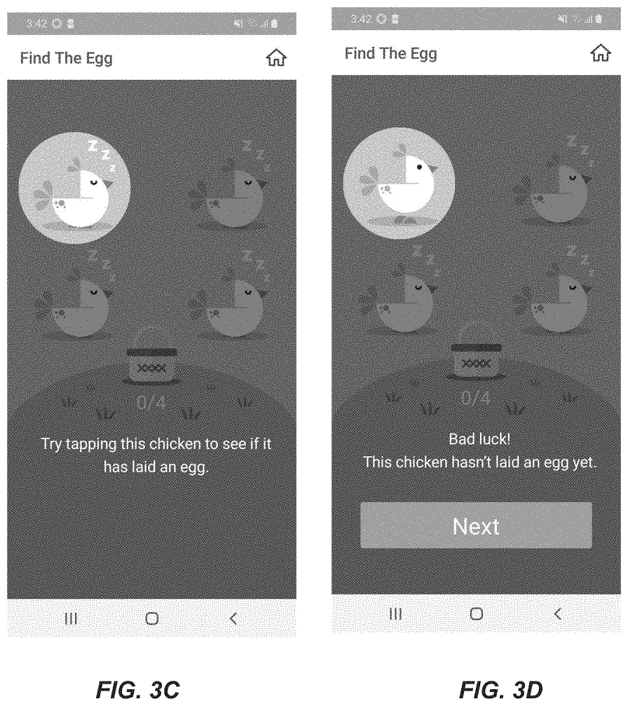

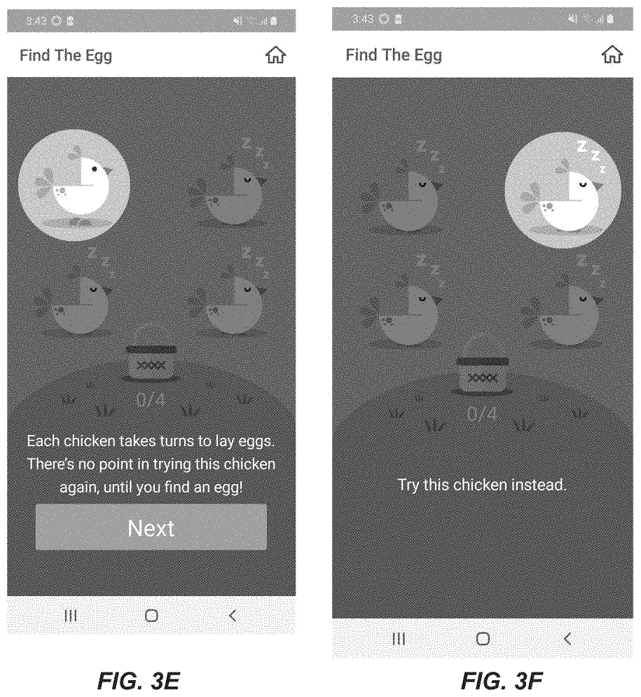

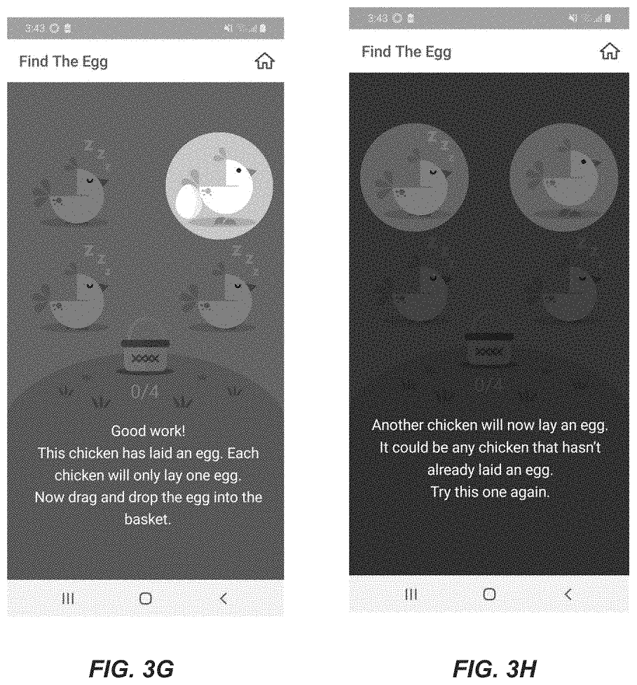

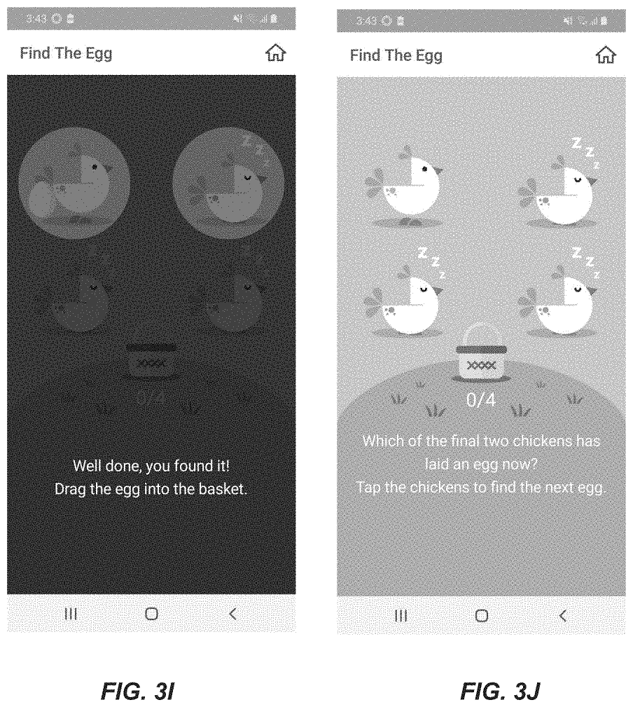

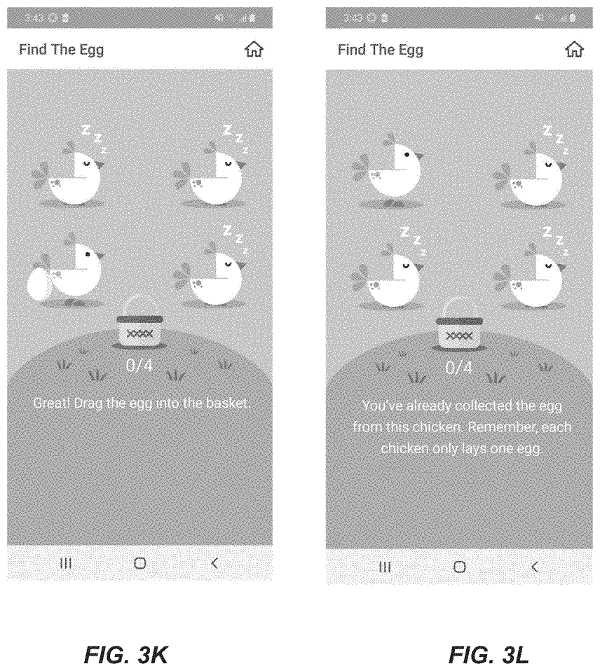

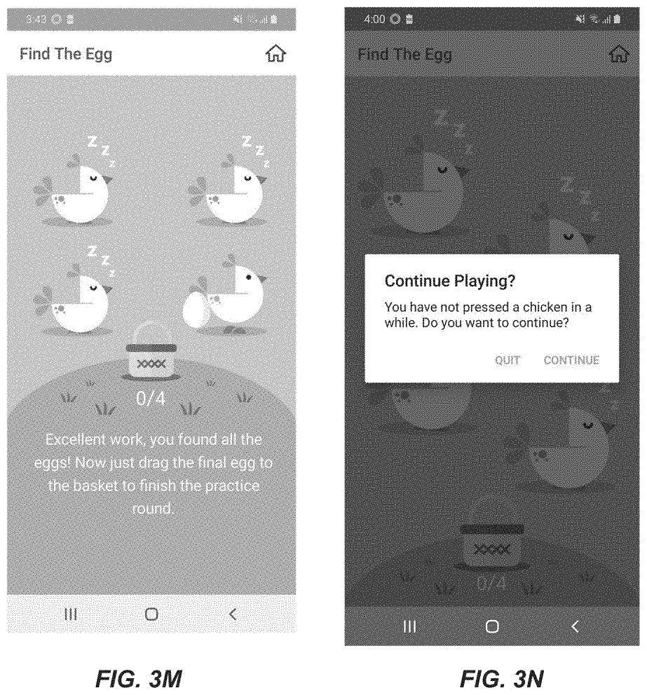

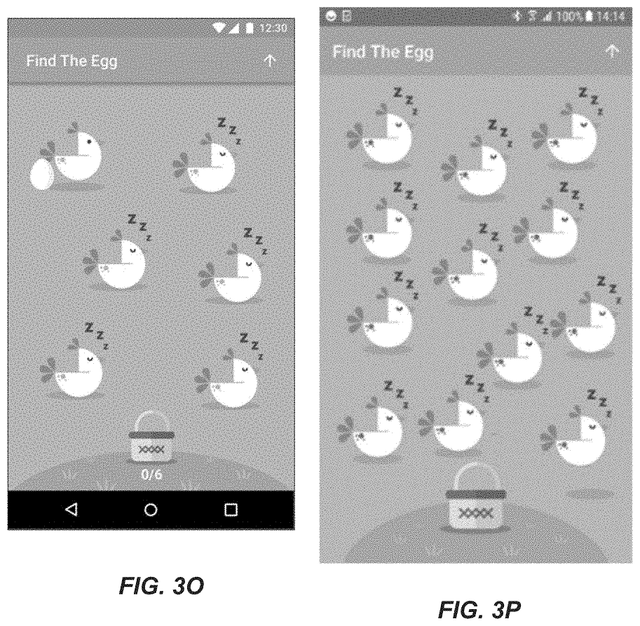

Tasks that may be performed using interactive games as described herein include identifying objects with particular properties from a set of objects. One such interactive game includes the “Find the Egg” task as described herein with respect to FIGS. 3A-3P. Two types of errors typically occur in the tasks described herein. Between-search errors (BSE) occur when an item that hid a target is selected more than once. Within-search errors (WSE) occur when an empty item (that did not contain an item before) is selected more than once. Note that after a target has been found, the set of items that could produce a WSE is reset.

In some instances, graphical user interfaces for interactive games may be generated and presented by a mobile computing device, such as device 107, based on the mobile computing device's execution of the software tool. In using the “Find the Egg” software tool, a patient (e.g., a user of the tool and/or the mobile computing device executing the tool) may find eggs by tapping on each illustrated chicken. The patient must remember where eggs were—and were not—found. The software tool implements certain rules to assess whether the patient commits certain errors. For example, each chicken might lay only one egg per game round, which may allow for the possibility for a user to commit between-search errors. In addition, only one egg might be laid at any one time, which may allow for the possibility for a user to commit within-search errors. Patients are instructed to find eggs by tapping on the chickens on the screen. To tap twice on a chicken that laid an egg before is a (between search) error. To tap twice on a chicken without eggs is a (within search) error. Once an egg has been found, another chicken lays a new egg. The difficulty of the task may change as users complete a round.

FIGS. 3A-3P depict example graphical user interfaces for an interactive game (e.g. the “Find the Egg” game) according to one or more illustrative aspects described herein. In FIG. 3A, a welcome screen according to one or more illustrative aspects is shown. The welcome screen provides initial instructions indicating that each chicken lays an egg and each chicken only lays one egg. The welcome screen also includes a first button that allow the user to obtain instruction on how to play the “Find the Egg” game and a second button that allows the user to begin playing the interactive game.

FIG. 3B shows a first instruction screen according to one or more illustrative aspects. The first instruction screen includes four chickens, a basket, a count of the number of eggs that have been found, and an instruction stating that each chicken will lay an egg and the user's task is to find all of the eggs. The first instruction screen also includes a button that takes the user to the next screen of the interactive game. FIG. 3C shows a second instruction screen according to one or more illustrative aspects. In the second instruction screen, a game board with a 2×2 array of chickens is provided. The top-left chicken is highlighted and a prompt is provided inviting the user to tap on the highlighted chicken to see if the chicken has laid an egg. FIG. 3D shows a third instruction screen according to one or more illustrative aspects. In the third instruction screen, a prompt is provided indicating that the top-left chicken (still highlighted) has not yet laid an egg. The third instruction screen also provides a button to take the user to the next screen. FIG. 3E shows a fourth instruction screen according to one or more illustrative aspects. In the fourth instruction screen, an instruction is provided indicating that each chicken takes turns to lay an egg. That is, only one chicken per round lays an egg. Therefore, the user should not interact with the top-left chicken until the user finds another chicken on the game board that has laid an egg. FIG. 3F shows a fifth instruction screen according to one or more illustrative aspects. In the fifth instruction screen, the top-right chicken in the game board has been highlighted. Additionally, a prompt is providing inviting the user to tap on the highlighted top-right chicken. FIG. 3G shows a sixth instruction screen according to one or more illustrative aspects. In the sixth instruction screen, an indication that the top-right chicken has laid an egg in response to the user selecting the top-right chicken is provided. Additionally, a prompt is provided indicating that the user should drag and drop the egg into the basket on the game board.

FIG. 3H shows a seventh instruction screen according to one or more illustrative aspects. In the seventh instruction screen, the top-left chicken is again highlighted once the user drags the previously found egg into the basket. The seventh instruction screen provides a prompt indicating that a second chicken has laid an egg and that the second chicken may be any chicken on the game board that has not already laid an egg. In the seventh instruction screen, those chickens include the top-left chicken, the bottom-left chicken, and the bottom-right chicken. FIG. 3I shows an eighth instruction screen according to one or more illustrative aspects. In the eighth instruction screen, an indication that the second egg was located under the top-left chicken is provided along with an instruction to drag the egg into the basket. FIG. 3J shows a ninth instruction screen according to one or more illustrative aspects. In the ninth instruction screen, an indication that the final two eggs are located under the chickens in the game board is provided. The ninth instruction screen also provides an indication that the user should select the chickens to find the next egg.

FIG. 3K shows a first game screen according to one or more illustrative aspects. The first game screen indicates that the user selected the lower-left chicken and the lower-left chicken had an egg under it. The first game screen also provides an indication that the user should drag the newly found egg into the basket. FIG. 3L shows a second game screen according to one or more illustrative aspects. In the second game screen, the user selected either the upper-left chicken or the upper-right chicken. The second game screen indicates that the user selected a chicken from which the user has already collected an egg along with providing a reminder that each chicken only lays one egg per game round. FIG. 3M shows a third game screen according to one or more illustrative aspects. The third game screen indicates that the user has selected the lower-right chicken and that the last egg was located under the lower-right chicken. The third game screen also provides an indication that the user should drag the last egg into the basket to finish the round.

FIG. 3N shows a timeout screen according to one or more illustrative aspects. In many embodiments, the interactive game tracks the user's usage of the mobile device during the game playing task. If the user has not provided any input within a threshold period of time, a prompt may be provided inviting the user to continue playing the game. The user may additionally elect to quit the game via the prompt.

FIG. 3O shows a fourth game screen according to one or more illustrative aspects. The fourth game screen includes six chickens in a 2×3 array on the game board along with the basket to which the eggs are to be dragged. As shown in the 2×2 game boards and the 2×3 game boards, the chickens are generally arranged in rows in columns that are evenly spaced within the display area of the mobile device providing the interactive game. FIG. 3P shows a fifth game screen according to one or more illustrative aspects. The fifth game screen includes twelve chickens in a 3×4 array. However, in the fifth game screen, the locations of the chickens are not arranged in rows in columns, particularly in the lower-right quadrant of the display area. In the fifth game screen, the location of the chickens has been modified to increase the difficulty of the interactive game and/or to better fit the chickens (e.g. interactive elements) within the display area of the mobile device. In this way, the game board for the interactive game may be dynamically generated based on the user's performance and/or the characteristics of the mobile device providing the interactive game.

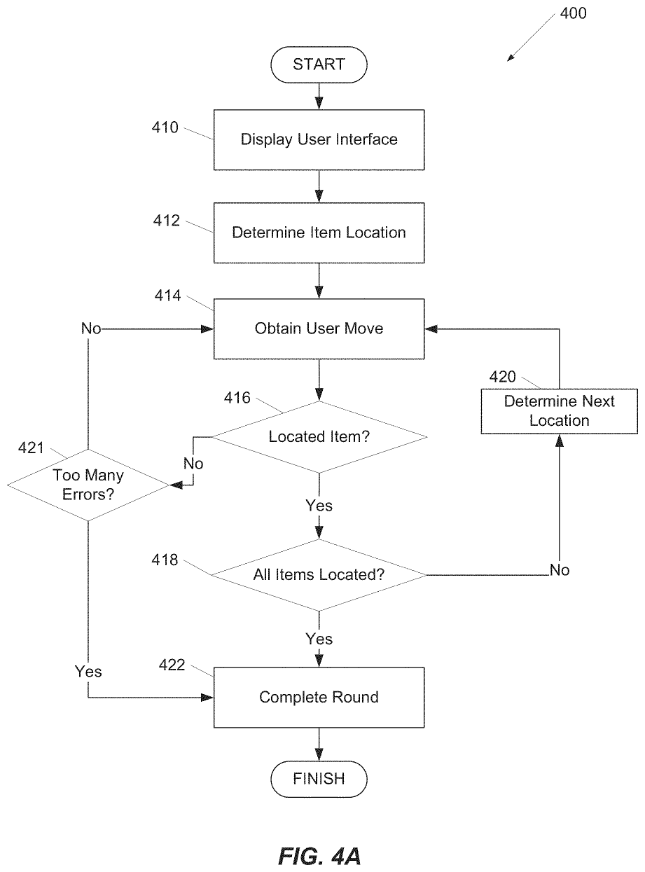

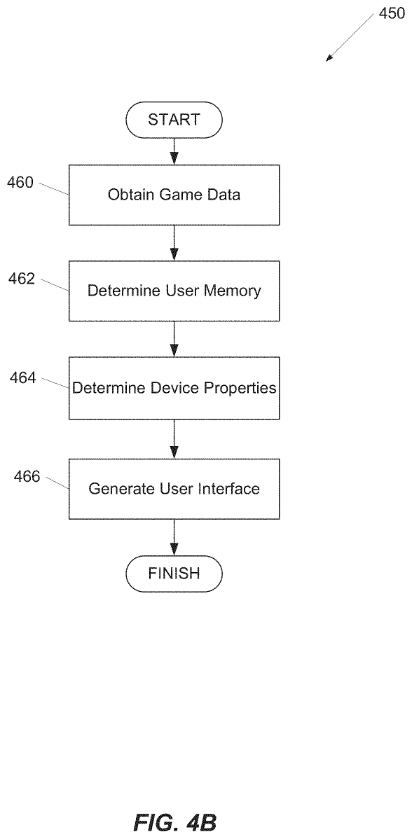

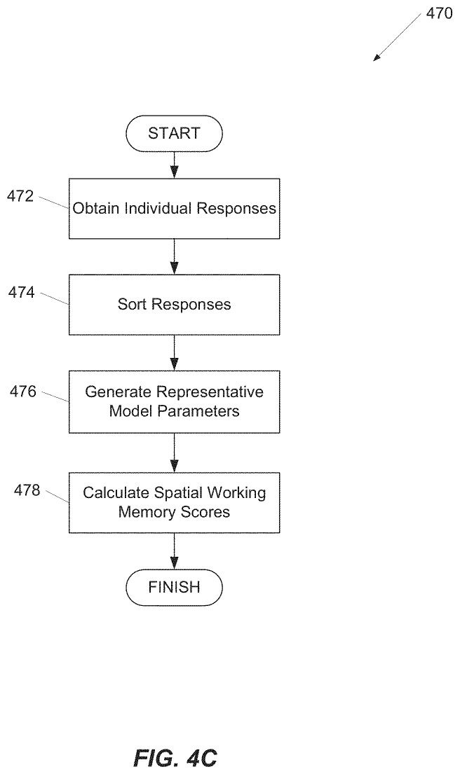

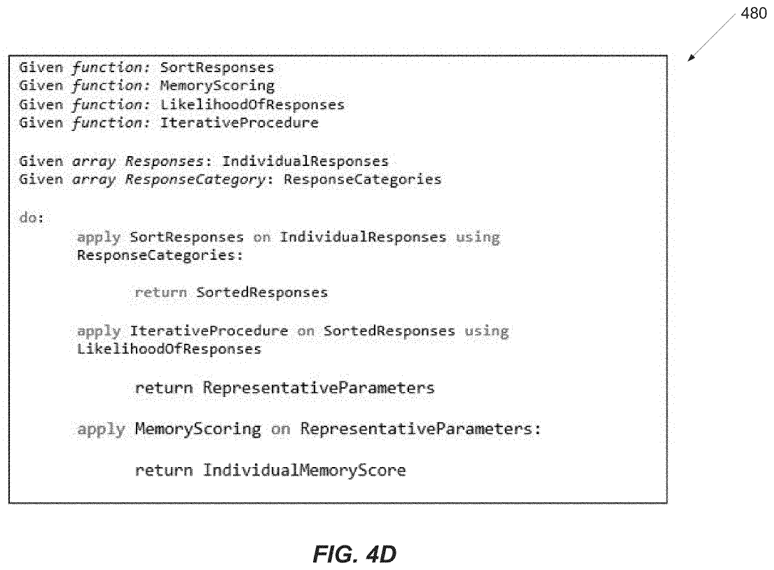

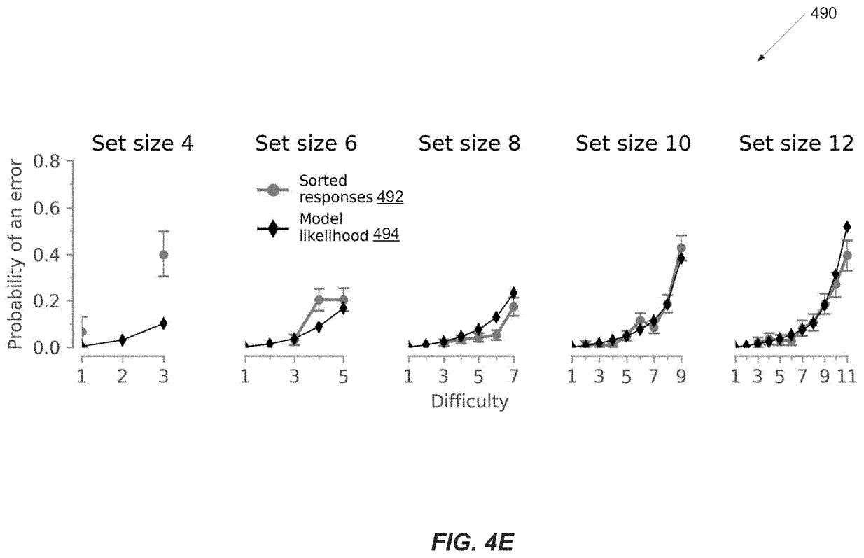

FIG. 4A is a flowchart conceptually illustrating a process for using a software tool according to one or more illustrative aspects described herein. Some or all of the steps of process 400 may be performed using one or more computing devices as described herein. In a variety of embodiments, some or all of the steps described below may be combined and/or divided into sub-steps as appropriate.

At step 410, a user interface may be displayed. The user interface may be for an interactive game. The user interface may include a game board having multiple interactive elements, such as the “Find the Egg” interactive game as described herein. The user interface may be displayed using a display of a mobile device providing the interactive game as described herein. In a variety of embodiments, the user interface may be dynamically generated based on the characteristics of the user and/or the mobile device as described in more detail with respect to FIG. 4B.

At step 412, an item location may be determined. The item location may correspond to one of the interactive elements in the game board. The item location may be an item to be found by the user. For example, in the “Find the Egg” interactive game, the item location may be a chicken and the item may be the egg. The item location may be determined randomly and/or predetermined as appropriate. In several embodiments, the item location is generated based on a previous round of the interactive game such that the item location does not correspond to a location of an item in the previous round. For example, if the previous round had a 2×2 game board and the items were located in the top-left, bottom-right, bottom-left, and top-right respectively, the first item location for the current round may be any location on the 2×2 game board except for the top-left. In this way, repetitive patterns in the game play over multiple rounds may be avoided.

At step 414, a user move may be obtained. The user move may be obtained using a touch screen of the mobile device. However, it should be noted that the user move may be obtained using any input device and/or network connection as described herein. The user move may indicate one of the interactive elements on the game board.

At step 416, if an item is located, the user may be prompted to drag the located item to a goal location on the game board. Once the user successfully locates an item in the game board, the process moves to step 418. If an item is not located, the process may move to step 421. A message may be displayed indicating that the user did not locate an item on the game board. When the user selects a location that the user previously selected, the user may be provided with an indication that the user already selected that location and that an item will not appear there until a later turn and/or an item has already been found at that location as appropriate. If a threshold number of incorrect selections have not been made at step 421, the process returns to step 414 to obtain another user move. In many embodiments, the number of incorrect selections by the user may be tracked and, if the user exceeds a threshold number of incorrect selections at step 421, the process may proceed to step 422.

At step 418, if all items have been located, the process moves to step 422. If not all items have been located, the process continues to step 420. At step 420, a next location may be determined. The next location may include any interactive element on the game board that has not previously been selected as a location. In this way, each interactive element on the game board may be associated with an item to be located by the user one time per game round. However, it should be noted that not every interactive element need be associated with an item during a round and/or an interactive element may be associated with an item more than once as appropriate. Once the next location has been determined, the process may return to step 414 to obtain the next user move.

At step 422, a round may be completed. Completing a round may display a summary screen showing the total number of moves by the user, the number of items found, the number of incorrect moves by the user, the time it took the user to complete the round, the current number of round and/or the total number of rounds to be played, and/or any other relevant information. A spatial working memory score may be calculated for the user based on any of the data generated during the round (and/or multiple rounds) as described herein and included in the summary screen. The summary screen may allow the user to proceed to the next round and/or complete the task associated with the interactive game as appropriate. A game session may include one or more rounds of the interactive game played by the user.

The adaptive design of the interactive games as described herein keeps the task interesting for the user, improves compliance, and reduces ceiling effects. However, each user has a different number and type of trials that are determined based on the skill of the user and/or capabilities of the user's mobile device. The skill of the user may correspond to the spatial working memory model for the user as described herein.

FIG. 4B is a flowchart conceptually illustrating a process for dynamically generating a user interface according to one or more illustrative aspects described herein. Some or all of the steps of process 450 may be performed using one or more computing devices as described herein. In a variety of embodiments, some or all of the steps described below may be combined and/or divided into sub-steps as appropriate.

At step 460, game data may be obtained. The game data may include one or more rounds of a current session of an interactive game, where each round includes a game board including a particular number of interactive elements as described herein. The game data for a round may also include moves made by the user during the round. The game data may further include a set of historical rounds played by the user. A variety of data describing the user, such as the user's age, IQ level, and the like, may also be included in the game data.

Data collected from the game may be further analyzed using mathematical models to generate scores that describe patients working memory and that may be further used to measure cognitive ability, to detect subgroups in clinical and non-clinical populations, identify changes caused by pharmacological and non-pharmacological interventions, track natural changes in cognitive ability over time and in general to characterize patients spatial working memory.

A variety of studies have provided data from interactive games, such as “Find the Egg,” played using mobile device tests administered to a set of study patients (e.g. users). During their first visit, a study nurse instructed patients about the protocol and provided them with a mobile device. According to the experimental schedule, the task was administered every fourth day. Items (e.g. chickens) were presented in the interactive game with sets of size 4, 6, 8, 10, or 12. Patients were instructed to find the hidden targets (e.g. eggs) by tapping on the items. Once a target was discovered, subjects were instructed to move the target to a basket in the bottom of the screen. A new search started by spawning a target below an item with the constraints that a) only one target was hidden at any given time and b) an item could only hide one target per block. Patients never received explicit feedback regarding their performance. Sessions included several blocks administered in succession. A block was completed once all the targets were found, in which case the set size was increased by two. A session was stopped when patients completed a block of set size 12 or when more than three between search errors were committed in a row. The difficulty of the task was adjusted across days by reducing the set size achieved during the previous session by two items, except for set size 4, in which case the difficulty stayed constant. This task design was employed to keep patients engaged and to reduce floor and ceiling effects.

A variety of probabilistic models may be used to measure SWM. These models quantify the probability that different moves (e.g., tapping an item) are done in interactive tasks as described herein. For example, in the first instantiation of such a model, all items tapped by the patients during the task may be stored jointly in SWM and therefore these items compete for the same pool of resources, i.e., memory slots. Therefore, it may be assumed that all items are stored in a fixed number of slots, called the memory pool. The number of available slots is referred to as the memory capacity M. It may be further assumed that items are stored in memory as long as there is capacity available, that items are stored and retrieved deterministically from memory and that only items that are not stored in memory are explored (i.e., are tapped within the interactive game)—these items may be referred to as the decision pool. Users may select items from the decision pool randomly.

From the assumptions above, errors are only possible when the number of previously tapped items that need to be stored in memory D (for difficulty), is larger than the memory capacity M. When D is larger than M, the probability of an error is the probability of selecting an item in the decision pool that has not been previously selected. The size of the decision pool is the set size S minus the memory capacity M. Thus, it may be written that (where P stands for probability and the random variables to the right of are the conditioning set):

$$P ( Error \vert D \leq M , S ) = 0 , P ( Error \vert D > M , S ) = \frac{D - M}{S - M} .$$

The parameter(s) describing the memory capacity of a patient is M. M may be estimated from patients' individual responses collected from the task described above. The assumption that only items in the decision pool are tapped may be relaxed by postulating that there is a small but positive probability η of selecting an item in the memory pool. This model parameter accounts for errors that the model cannot directly explain and that are caused by a variety of factors, such as attention slips.

The probabilities of between search errors (BSE) and within search errors (WSE) given a set of parameters and the state of the board in the game may be specified by adding further assumptions and these probabilities may be used to process the data obtained from the app. Between search items are defined as items on the screen (“chickens”) that had a target (“an egg”). Between search errors are defined as moves in which a patient taps on a between search item. Within search items are defined as items that have been tapped since the last target was found but were not between search items. Within search errors are defined as moves in which a within search item is tapped.

In several embodiments, these assumptions include that all items have the same probability of being left outside memory, i.e., all items are stored with the same priority. From this, the probability of a BSE is equal to the number of between search items B divided by the difficulty D:

$$P ( between search error \vert M , B , S , T ) = ( \frac{B}{D} ) \frac{D - M}{S - M} .$$

where the difficulty D is the sum of B and the number of within search items W. From there, it follows that:

$$P ( within search error \vert M , B , S , T ) = ( \frac{D - B}{D} ) \frac{D - M}{S - M} .$$

The model above predicates on the assumption that between-search and within-search items are treated equally in memory. This premise may be replaced by either that within-search items are given priority in memory and/or that between-search items are given priority. In the first case, between search items are put in memory only once all within search items have been stored. Thus, WSE are only possible when the number of within search item W exceeds the memory capacity M:

$$P ( within search error \vert M \geq W , D , S ) = 0 , P ( within search error \vert M < W , D , S ) = \frac{W - M}{T - M} .$$

This may be referred to as the within search priority model. The between search priority model is defined by the equations

$$P ( between search error \vert M \geq B , D , S ) = 0 , P ( between search error \vert M < B , D , S ) = \frac{B - M}{T - M}$$

The models above may also be modified by removing the assumption that a single memory storage is used to store between- and within-search items. This may be done because the demands imposed by the task on the recalling of each type of item are very different. Within-search items may need to be stored only shortly and are regularly removed from memory once a new target is discovered, while between search errors may be stored for longer periods and should not be overwritten across searches. If both types of items do not compete for the same memory slots, between- and within-search items may be stored in independent memory pools of capacity M_{B} and M_{W}.

As in the previous models, BSE are only possible when the number of between search items B exceeds the between search memory capacity M_{B}:

P (between search error|M_{B}≥B,D,S)=0.

In another case, the probability is given by:

$$P ( between search error \vert {M}_{B} , {M}_{W} , B , D , S ) = \frac{B - {M}_{B}}{S - {M}_{B} - min ( {M}_{W} , W )} .$$

The denominator corresponds to the size of the decision pool, which is the set size S minus the total number of items in any memory M_{B}+min(M_{W}, W). Also, it follows that

$$P ( within search error \vert {M}_{B} \geq B , D , S ) = 0. \\ P ( within search error \vert {M}_{B} , {M}_{W} , B , D , S ) = \frac{W - {M}_{w}}{S - {M}_{W} - min ( {M}_{B} , B )} .$$

The previous models are built upon the assumption that memory storage and retrieval are deterministic. This postulate may be relaxed in a number of ways. As before, it may be assumed that a number of items stored in memory are always correctly retrieved. The remaining between-search items are stored in memory but their retrieval is stochastic and fails with rate r, independently of the set size.

As with the previous models, in this model items that are correctly retrieved from memory are excluded from the decision pool. Formally, the between search memory may be divided into a deterministic storage with capacity M_{B} and a fuzzy storage with variable capacity M_{F}. The conditional probability of a BSE is given by

$$P ( between search error \vert {M}_{B} , {M}_{F} , {M}_{W} , B , D , S ) = \frac{B - {M}_{B} - {M}_{F}}{S - {M}_{B} - {M}_{F} - min ( {M}_{W} , W )} .$$

The probability of M_{F} correct retrievals depends on the failure rate r and the maximal capacity of the fuzzy memory component Mₘₐₓ.

$$P ( {M}_{F} \vert {M}_{max} , r ) = ( \begin{matrix}{M}_{max} \\ {M}_{F}\end{matrix} ) {{r}^{{M}_{max} - {M}_{F}} ( 1 - r )}^{{M}_{F}} .$$

The unused capacity may or may not be allocated to increase the precision of the memory capacity that is being used. If it is not allocated to other slots, the marginal probability of a BSE is given by

$${\sum }_{{M}_{F} = 0}^{{M}_{u s e d}} \frac{B - {M}_{B} - {M}_{F}}{T - {M}_{B} - {M}_{F} - min ( {M}_{w} , W )} ( \begin{matrix}{M}_{used} \\ {M}_{F}\end{matrix} ) {{r}^{{M}_{u s e d} - {M}_{F}} ( 1 - r )}^{{M}_{F}} , {M}_{used} = min ( B - {M}_{B} , {M}_{max} ) .$$

This formula corresponds to the marginal probability of a BSE for different memory sizes M_{B}+M_{F}, where M_{F} items fail at retrieval at rate r.

A cumulative working memory score may be computed, for example, as the sum of deterministic memory capacity and the expected number of items stored in fuzzy memory

Memory score=M_{B}+rMₘₐₓ

A practical limitation with Mₘₐₓ is that, unless the task is explicitly designed to challenge patients to difficulties in which B−M_{B}>Mₘₐₓ, it may not be uniquely identifiable. In practice, it may be assumed that

M_{used}=B−M_{b}.

This method is useful to estimate r even when the maximal set size is lower than the maximal memory capacity.

At step 462, user memory may be determined. The user memory may include a spatial working memory score for the user. The spatial working memory score may be calculated on a round-by-round and/or a per-session basis as appropriate. Any of a variety of techniques, including those described herein, may be used to determine the spatial working memory score for the user. In several embodiments, the user memory may be determined for the current session and/or one or more historical sessions. In a variety of embodiments, the user memory is determined based on one or more spatial working memory scores. For example, the user memory may be determined as a moving average of the spatial working memory scores for the user over a particular time frame and/or number of game sessions.

At step 464, device properties may be determined. The device properties may include any characteristics of the device on which the user interface will be displayed. These characteristics may include, but are not limited to, screen size, screen resolution, pixel density, input device type, operating system version, and the like. At step 466, a user interface may be generated. The user interface may be generated based on the determined user memory and/or the determined device properties. In this way, the user interface may be dynamically generated based on the relative skill of the user and the characteristics of the device being used to play the interactive game. The user interface may include a game board having a number of interactive elements and/or a layout of the interactive elements within the game board. The number of interactive elements may be determined based on the user memory and/or device properties. In a variety of embodiments, more interactive elements may be included in the game board as the user memory score increases. For example, if a user memory score ranges from 0-100, when the user memory score is between 0 and 25 the game board may include four interactive elements. When the user memory score is between 26 and 50, the game board may include six interactive elements, eight interactive elements when the user memory score is between 51 and 75, and twelve interactive elements when the user memory score is between 76 and 100. However, it should be noted that any number of interactive elements and/or user memory ratings may be used to determine the number of interactive elements as appropriate.

The layout of and/or size of the interactive elements on the game board may be determined based on the user memory and/or device properties. For example, if a device has a six-inch screen, the interactive elements may be ½ inch square, while the interactive elements may be ¼ inch square on a device having a four-inch screen. The size of the interactive elements may also be determined in pixels and based on the pixel density of the device such that the size of the interactive elements remains consistent across devices of varying screen sizes and/or resolutions. The size of the interactive elements may also be determined based on the number of interactive elements on the game board such that consistent amount of the game board is occupied by interactive elements for a particular round. Similarly, the location of the slots within the game board may be determined based on the device properties and/or the number of interactive elements. As described herein, the slots within the game board may be arranged in square rows and columns and/or dispersed unevenly throughout the game board. In several embodiments, the layout of the slots is determined based on the user memory. For example, when the user memory exceeds a threshold value, the layout of the game board may be such that some areas of the game board are more densely populated with interactive elements, thereby increasing the difficulty of remembering particular items within that portion of the game board. In this way, the number of confusable neighbors for a particular interactive element may be dynamically adjusted based on the user memory and/or device properties. The maximum density of interactive elements may be determined based on the type of input devices available, screen size, interactive element size, and/or padding sizes as appropriate. For example, more padding may be needed between interactive elements on a device having a touch screen than a device using a mouse as a touch screen is typically less accurate than a mouse. In this way, the layout of the interactive elements within the game board may allow for each interactive element to be selected with relative accuracy.

In several embodiments, the number and/or layout of interactive elements within the game board may be determined using one or more machine classifiers. It should be readily apparent to one having ordinary skill in the art that a variety of machine classifiers may be utilized including (but not limited to) decision trees, k-nearest neighbors, support vector machines (SVM), neural networks (NN), recurrent neural networks (RNN), convolutional neural networks (CNN), and/or probabilistic neural networks (PNN). RNNs may further include (but are not limited to) fully recurrent networks, Hopfield networks, Boltzmann machines, self-organizing maps, learning vector quantization, simple recurrent networks, echo state networks, long short-term memory networks, bi-directional RNNs, hierarchical RNNs, stochastic neural networks, and/or genetic scale RNNs.

Mathematical models in accordance with aspects of the disclosure may specify the probability of individual responses in the task depending on, but not limited to, the items that a patient has previously explored, the number of items on the screen, etc.

FIG. 4C is a flowchart conceptually illustrating a process for deriving working memory scores given individual data according to one or more illustrative aspects described herein. Some or all of the steps of process 470 may be performed using one or more computing devices as described herein. In a variety of embodiments, some or all of the steps described below may be combined and/or divided into sub-steps as appropriate.

At step 472, individual responses may be obtained. Individual response may be obtained by a mobile device providing an interactive game (e.g. “Find the Egg” or any other similar app or psychological test) to a patient. A data server may obtain individual response from one or more mobile devices. In many embodiments, individual responses include any items selected by the user in an individual screen and/or any associated information thereof, such as reaction time, number of items in the screen, items previously selected, etc.

At step 474, the individual responses may be sorted according to, for example, the number of items on the screen, the history of responses of that individual, the number of previous errors, the items previously found, etc. At step 476, the probability of the parameters, as defined by the model(s), may be calculated using the (sorted) responses. An iterative procedure based on the probability of the parameters and the sorted responses may be used to compute representative parameters. Iterative procedures include, but are not limited to, Markov Chain Monte Carlo simulations, grid search, Bayesian estimation, and/or a mixture of any of these methods or any other comparable methods. As shown in FIG. 4E, individual responses 490 from a single patient playing Find the Egg were sorted according to set size and difficulty. Using an iterative procedure, representative parameters were estimated by increasing the likelihood of the data (494) compared to the sorted individual responses (492). At step 478, spatial working memory scores may be computed as a mathematical combination of the said representative parameters as described herein. Pseudocode 480 for generating spatial working memory scores using iterative methods is shown in FIG. 4D. Any of a variety of computing devices as described herein can execute instructions embodying pseudocode 480 to perform any of a variety of processes as described herein.

A variety of patients may utilize mobile devices to play interactive games as described herein. In several embodiments, data from the variety of patients may be collected and analyzed to determine the effectiveness of treatments and/or determine potential treatments that may be administered to the patients.

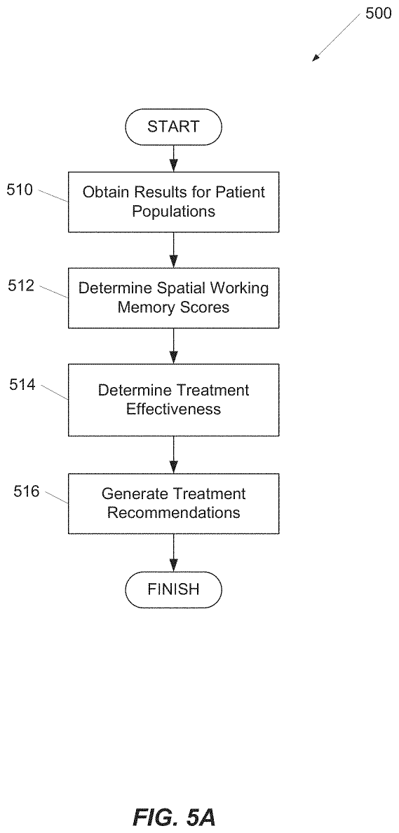

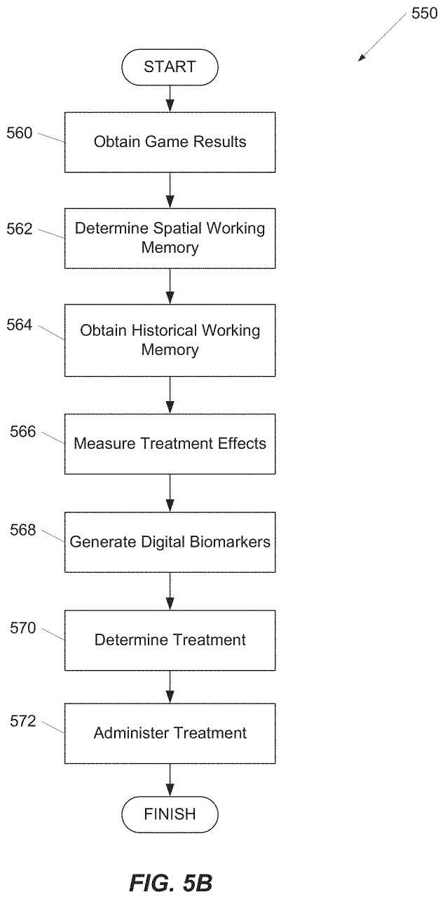

FIG. 5A is a flowchart conceptually illustrating a process for analyzing spatial working memory according to one or more illustrative aspects described herein. Some or all of the steps of process 500 may be performed using one or more computing devices as described herein. In a variety of embodiments, some or all of the steps described below may be combined and/or divided into sub-steps as appropriate.

At step 510, results for one or more patient populations may be obtained. The results may be obtained from a variety of sources, such as mobile devices used by the patients to play one or more interactive games and/or complete tasks as described herein. The results may include game boards and/or sets of moves for one or more sessions of an interactive game played by each patient as described herein. The game results may include one or more historical game sessions played by the patient. The game results may also include a variety of characteristics for each patient, such as the user's age, gender, IQ level, and the like. The patients of a particular patient population may have one or more characteristics in common. These characteristics may include, but are not limited to, age, current conditions, diseases, mental capacity, cognitive ability, SWM scores, administered treatments, and the like. For example, members of a first patient population may have ASD while members of a second patient population may have Alzheimer's. In another example, patients in a first patient population may have ASD and be under the age of 12, while patients in a second patient population also have ASD but are over the age of 12. In a third example, patients in a first patient population may be prescribed a first treatment, while patients in a second patient population may be prescribed a second treatment. However, it should be noted that any number of patient populations may be used and/or any particular combination of characteristics may be used to determine the patient populations. In this way, patient populations may include one or more patients having common characteristics such that patients in different populations may be analyzed and/or compared as appropriate.

At step 512, spatial working memory scores may be determined. The spatial working memory scores may be determined for each of the patients in the patient populations using any of a variety of mathematical models as described herein. In a number of embodiments, the spatial working memory scores for a particular patient includes a time series analysis for the patient based on multiple game sessions played by the user. The spatial working memory scores for a patient may include a current spatial working memory score determined based on the last session played by the user and/or multiple scores for different game sessions over time. In many embodiments, the time series of analysis for the patient is based on the number of correct moves provided by the user in one or more game sessions and/or the amount of time that it takes the player to play one or more rounds of the interactive game in the game session(s). In several embodiments, the time series analysis is based on the number of incorrect moves provided by the user in one or more game sessions and/or the amount of time that it takes the player to play one or more rounds of the interactive game in the game session(s). For example, time series analysis may be based on how the patient's gameplay choices change (or do not change) from one game session to the next. This is due to ASD patients potentially making the same mistakes as the patients may not learn from one game session to the next such that the patients perform the same memory retrieval process each game session to arrive at the same result.

At step 514, treatment effectiveness may be determined. The treatment effectiveness may be determined on a per-patient basis and/or on a per-patient population basis as appropriate. The treatment effectiveness for a particular patient may be determined for a patient based on the spatial working memory scores for the patient. The treatment effectiveness for a patient population may be determined based on the spatial working memory scores for each patient in the patient population. Any of a variety of statistical techniques may be used to determine treatment effectiveness as appropriate. For example, the current spatial working memory scores for the patient population may be compared to one or more historical spatial working memory scores for the patient population. The delta between the current spatial working memory score and one or more of the historical spatial working memory scores, a rate of change over time, a moving average of the spatial working memory scores, and/or any other statistical measure may be used to evaluate the treatment effectiveness. The treatment effectiveness may also be compared across patient populations. For example, a drug may be administered to patient populations divided by age to determine the effectiveness of the drug on improving spatial working memory scores for the patient based on the age of the patient. In a second example, a first patient population may be administered a first drug and a second patient population may be administered a placebo. The difference change in spatial working memory scores between the first patient population and the second patient population may be used to determine the effectiveness of treatments including the first drug. In a third example, a first patient population may be administered a first drug and a second patient population may be administered a second drug. The difference change in spatial working memory scores between the first patient population and the second patient population may be used to determine which of the first drug and the second drug are more effective at improving spatial working memory. In a fourth example, a first patient population may be administered a drug at a first dosage and a second patient population may be administered the same drug at a second dosage. The difference in improvement (or lack thereof) in spatial working memory for the patient populations may be used to determine the effectiveness of particular dosages of the drug. However, it should be noted that any comparison, such as between patient populations having different conditions affecting spatial working memory, may be used to determine treatment effectiveness as appropriate.

At step 516, treatment recommendations may be generated. The treatment recommendations may be generated based on the effectiveness of the treatments and/or the characteristics of particular patient populations. The treatment recommendations may be generalized and/or targeted towards patients having particular characteristics in common with particular patient populations. For example, if a particular drug is effective in improving spatial working memory for ASD patients under the age of 12, the generated treatment recommendation may include a particular dosage for the drug for ASD patients under the age of 12. Similarly, if the particular drug is not effective in improving spatial working memory for ASD patients over the age of 12 and/or for Alzheimer's patients, the treatment recommendation may not include the drug for patients having these characteristics. The treatment recommendations generated for patient populations may be used to recommend and/or administer treatments to particular patients having characteristics in common with the patient populations as described herein.

FIG. 5B is a flowchart conceptually illustrating a process for administering treatments according to one or more illustrative aspects described herein. Some or all of the steps of process 550 may be performed using one or more computing devices as described herein. In a variety of embodiments, some or all of the steps described below may be combined and/or divided into sub-steps as appropriate.

At step 560, game results may be obtained. The game results may include game boards and/or user moves for one or more current sessions of an interactive game as described herein. The game results may include one or more historical game sessions played by the user. The game results may also include a variety of characteristics of the user, such as the user's age, gender, IQ level, and the like. At step 562, spatial working memory may be determined using mathematical model explained herein. The spatial working memory may be determined for current sessions played by the user as described herein. At step 564, historical spatial working memory may be obtained. The historical spatial working memory may be determined for one or more historical session played by the user as described herein.

At step 566, treatment effects may be measured. The treatment effects may be measured based on the spatial working memory score and/or the historical spatial working memory scores or any other clinically relevant method. For example, the spatial working memory score for the current game session may be compared to one or more historical spatial working memory scores for the user determined in previous game sessions. The measured treatment effects may include the delta between the current spatial working memory score and one or more of the historical spatial working memory scores, a rate of change over time, a moving average of the spatial working memory scores, and/or any other statistical measure as appropriate.

At step 568, a digital biomarker indicating a particular patient (or class of patient) can be generated based on the task results and/or the SWM scores for the patient. A task difficulty level can be determined for a task for assessment of a patient having a neurological condition as described herein. An interactive task at the task difficulty level can be generated for the patient as described herein. A user interface for a mobile device and for receiving task input from the patient attempting to complete the task can be generated as described herein. A task outcome can be determined based on the received task input as described herein. A modified task difficultly level based on the received task input as described herein. Task outcome steps can be iteratively generated using the modified task difficulty level until a predetermined condition is met as described herein. A digital biomarker can be determined for the patient by analyzing the plurality of received task inputs and determined task outcomes as described herein.

The predetermined condition may include the patient completing the task at a predetermined difficulty level. The predetermined condition may include the patient making at least a predetermined number of errors while attempting to complete any task. An interactive task can include hiding an object on a game board as described herein. Each difficulty can be associated with a different number of interactive elements on the game board as described herein. The digital biomarker may be determined any computing device, such as a server device, configured to receive task input and task outcomes. The patient may selected from a population that has been administered a treatment as described herein. A historical digital biomarker may be obtained for each patient in the in the population as described herein. A digital biomarker may be generated for each patient in the population as described herein. The historical digital biomarker and the new digital biomarker for each patient may be compared to determine a treatment effectiveness for the administered treatment as described herein. Generating a modified task difficulty level may include using an iterative procedure such as Markov Chain Monte Carlo simulations, grid search, and Bayesian estimation as described herein.

At step 570, a treatment may be determined. Treatments may include pharmaceuticals, such as Balovaptan and/or Arbaclofen as well as particular tasks designed to improve a user's performance. In a variety of embodiments, the determined treatment includes Vasopressin 1a antagonist, more particularly Arbaclofen, Balovaptan, a GABA-Aa5 PAM, a GABA-A1 modulator, a mGlu4/7 PAM, a Dopamine 2 receptor antagonist, in particular Risperidone, mu-opioid receptor antagonist, in particular naloxone, and/or NMDA glutamate receptor antagonist, in particular memantine, and pharmaceutically acceptable salts thereof.

The treatment may be determined based on the user's digital biomarker, characteristics, measured treatment effects, and/or the user's current spatial working memory score. For example, if the user has been taking a particular dose of Balovaptan and the user's spatial working memory score has not been improving, an alternative treatment (such as arbaclofen) may be determined. The dosage of a particular pharmaceutical may be determined to be a therapeutically effective dose based on the user's spatial working memory score and/or desired improvement in spatial working memory score. In a second example, if the user is experiencing a decrease in spatial working memory scores as the difficulty in assigned tasks increases, the determined treatment may include providing additional simpler tasks to the user in order to build up the user's confidence in performing the more difficult tasks. Similarly, other tasks and/or treatments may be determined to be useful to the user in order to increase their spatial working memory. For example, if the user is not experiencing a sufficient amount of improvement in their spatial working memory score determined based on playing the “Find the Egg” interactive game, the user may be prescribed different interactive tasks that exercise their spatial working memory in a different manner. In another example, if the current spatial working memory score is over a threshold value and an improvement over a historical spatial working memory score, the dosage of a particular drug may be reduced in order to maintain the user's current spatial working memory score without over-medicating the user.

At step 572, a treatment may be administered. The provided treatment may be determined based on the digital biomarker, measured treatment effects, and/or determined treatment as appropriate. The administered treatment may include a therapeutically effective dose of a drug to improve the spatial working memory score for the user. For example, a therapeutically effective dose of Balovaptan and/or Arbaclofen may be administered to the user. However, it should be noted that any appropriate drug, such as Vasopressin 1a antagonist, more particularly Arbaclofen, Balovaptan, a GABA-Aa5 PAM, a GABA-A1 modulator, a mGlu4/7 PAM, a Dopamine 2 receptor antagonist, in particular Risperidone, mu-opioid receptor antagonist, in particular naloxone, and/or NMDA glutamate receptor antagonist, in particular memantine, and pharmaceutically acceptable salts thereof may be used as appropriate. Similarly, particular interactive tasks may be provided to the user to complete in accordance with the determined treatment.

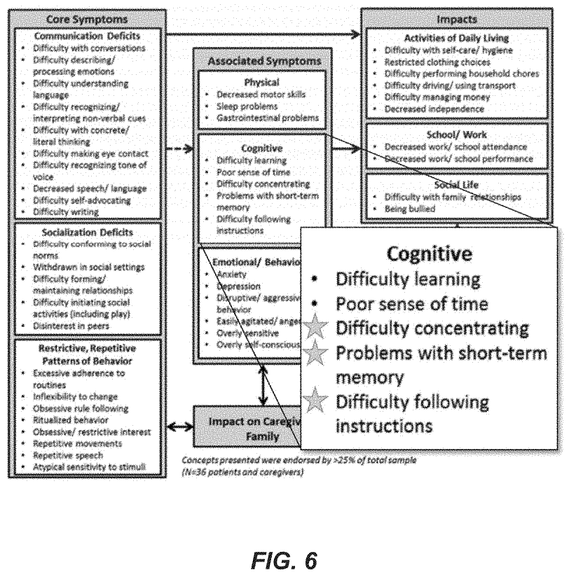

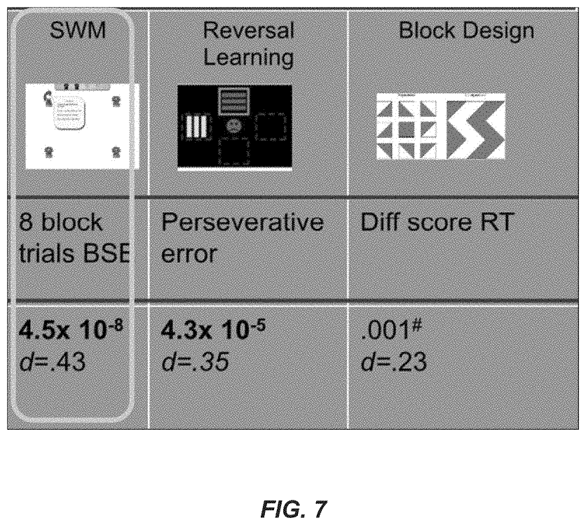

One or more aspects of the disclosure may implement one or more assessments that focus on spatial working memory (SWM) for one or more reasons. For example, SWM may plausibly underlie meaningful challenges for autistic individuals. Examples of one or more these challenges are illustrated in FIG. 6. Additionally or alternatively, neuropsychological tests of SWM may show robust effect sizes in case/control comparisons. Examples of these effect sizes are illustrated in FIG. 7.

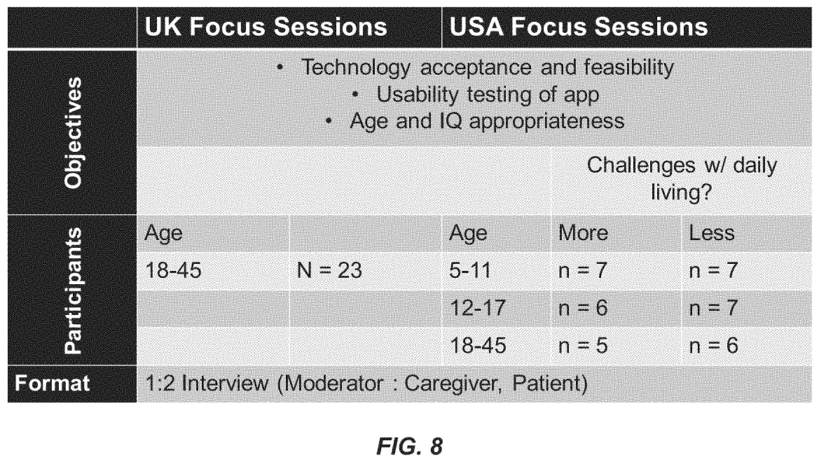

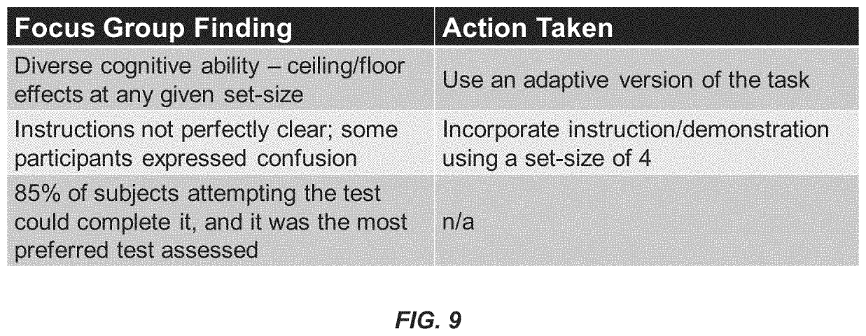

Some reactions from the ASD Community to the prototype associated with one or more aspects of the disclosure have been captured through experimental testing with two focus groups including autistic individuals (and their caregivers as available). Details about the objectives, patients, and format of the focus groups are illustrated in FIG. 8. In addition, findings from the focus groups and actions taken in view of these findings are illustrated in FIG. 9. Subsequently, one or more agent-based simulations were performed by the focus groups. In particular, before deploying the task in an empirical validation study, an attempt was made to understand and/or confirm how the task's design might influence its sensitivity. Specifically, termination rules were examined to determine whether error types depend on whether the task terminates after a particular number of errors (e.g., 3 errors, 5 errors, 7 errors, etc.) are made. In addition, set sizes were examined to determine whether the number of interactive elements (e.g. chickens) affects the relative pattern of errors. Spatial working memory capacity was examined to determine whether the effect of termination rule and set-size on error types itself depends on spatial working memory capacity. To address these questions, a computational model capable of performing the task was made, under several key assumptions. First, subjects try to remember previously searched locations of two types: searched locations where an egg was found and searched locations where an egg was not found. Second, if the memoranda exceed working memory capacity, the oldest items are forgotten. Third, crowding and strategic “inversion” effects do not play a major role in the user's performance.

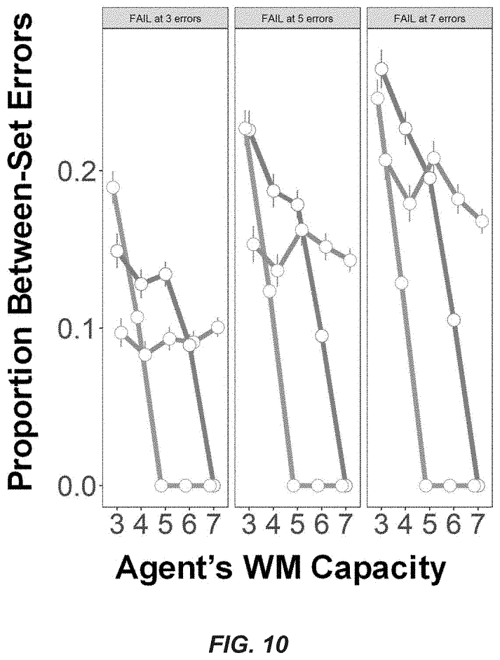

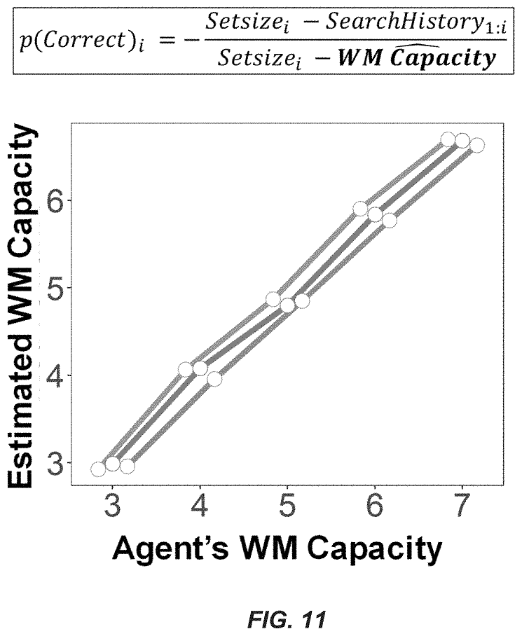

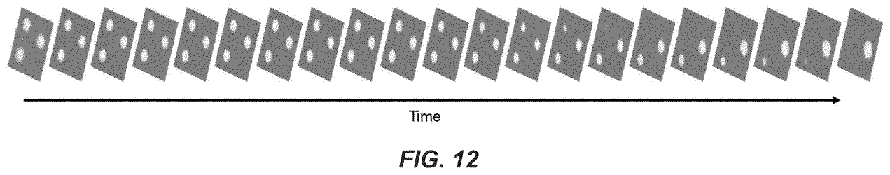

The computational model yielded several results relevant to how the task's design might affect its sensitivity, particularly with respect to the termination rule, set size, and spatial working memory capacity questions noted above; these results are illustrated in FIGS. 10 and 11. As seen in model results illustrated in FIGS. 10 and 11, regardless of the termination rule, between-set errors may strongly differentiate the users according to their SWM capacity. In addition, regardless of the termination rule, SWM capacity may be expressed as a function of probability of trial-by-trial searching, given trial history. Therefore, to minimize patient burden, the task may be deployed with a termination rule of three errors and an adaptive set-size, determined on a per-subject basis.

Neural extensions to the model associated with one or more aspects of the disclosure may be able to characterize spatial crowding effects as a function of excitatory-inhibitory balance, as illustrated in FIG. 12. As seen in FIG. 12, the neural model shows the firing rate (e.g., dots are representative of a higher rate) of a 35×35 excitatory point neuron array after a 3-location stimulus is presented. The model and its associated neural extensions may be implemented using one or more of a variety of machine classifiers as described herein. Forgetting (right side) arises through attractor decay. By modulating the excitatory and inhibitory conductance, the model aims to predict the spatial errors under GABA modulation. GABA modulation may be affected by the administration of a variety of pharmaceuticals, such as arbaclofen.

Although the present invention has been described in certain specific aspects, many additional modifications and variations would be apparent to those skilled in the art. In particular, any of the various processes described above may be performed in alternative sequences and/or in parallel (on different computing devices) in order to achieve similar results in a manner that is more appropriate to the requirements of a specific application. It is therefore to be understood that the present invention may be practiced otherwise than specifically described without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A computer-implemented method of generating a digital biomarker, comprising:
determining a task difficulty level for assessment of a patient having a neurological condition;
generating an interactive task at the task difficulty level;
generating for display on a mobile device a graphical user interface for receiving task input from the patient attempting to complete the task;
determining a task outcome based on the received task input;
generating a modified task difficultly level based on the received task input;
iterating through the previous generating and determining the task outcome steps using the modified task difficulty level until a predetermined condition is met; and
based on the predetermined condition being met, determining a digital biomarker for the patient by analyzing the plurality of received task inputs and determined task outcomes.

2. The computer-implemented method of claim 1, wherein the predetermined condition comprises the patient completing the task at a predetermined difficulty level.

3. The computer-implemented method of claim 1, wherein the predetermined condition comprises the patient making at least a predetermined number of errors while attempting to complete any task.

4. The computer-implemented method of claim 1, wherein the interactive task comprises hiding an object on a game board, and wherein each difficulty is associated with a different number of interactive elements on the game board.

5. The computer-implemented method of claim 1, wherein determining the digital biomarker is performed by a server device configured to receive task input and task outcomes.

6. The computer-implemented method of claim 1, wherein:
the patient is selected from a population that has been administered a treatment; and
the method further comprises:
obtaining a historical digital biomarker for each patient in the in the population;
generating a new digital biomarker, using the method of claim 1, for each patient in the population; and
determining, based on a comparison of the historical digital biomarker and the new digital biomarker for each patient, a treatment effectiveness for the administered treatment.

7. The computer-implemented method of claim 1, wherein generating the modified task difficulty level comprises using an iterative procedure selected from the group consisting of Markov Chain Monte Carlo simulations, grid search, and Bayesian estimation.

8. A non-transitory machine-readable medium storing instructions that, when executed by one or more processors, cause the one or more processors to perform steps comprising:
determining a task difficulty level for assessment of a patient having a neurological condition;
generating an interactive task at the task difficulty level;
generating for display on a mobile device a graphical user interface for receiving task input from the patient attempting to complete the task;
determining a task outcome based on the received task input;
generating a modified task difficultly level based on the received task input;
iterating through the previous generating and determining the task outcome steps using the modified task difficulty level until a predetermined condition is met; and
based on the predetermined condition being met, determining a digital biomarker for the patient by analyzing the plurality of received task inputs and determined task outcomes.

9. The non-transitory machine-readable medium of claim 8, wherein the predetermined condition comprises the patient completing the task at a predetermined difficulty level.

10. The non-transitory machine-readable medium of claim 8, wherein the predetermined condition comprises the patient making at least a predetermined number of errors while attempting to complete any task.

11. The non-transitory machine-readable medium of claim 8, wherein the interactive task comprises hiding an object on a game board, and wherein each difficulty is associated with a different number of interactive elements on the game board.

12. The non-transitory machine-readable medium of claim 8, wherein determining the digital biomarker is performed by a server device configured to receive task input and task outcomes.

13. The non-transitory machine-readable medium of claim 8, wherein the instructions, when executed by one or more processors, further cause the one or more processors to perform steps comprising:
obtaining a historical digital biomarker for each patient in the in a population, wherein each patient selected from the population has been administered a treatment;
generating a new digital biomarker, using the previously recited steps, for each patient in the population; and
determining, based on a comparison of the historical digital biomarker and the new digital biomarker for each patient, a treatment effectiveness for the administered treatment.

14. The non-transitory machine-readable medium of claim 8, wherein generating the modified task difficulty level comprises using an iterative procedure selected from the group consisting of Markov Chain Monte Carlo simulations, grid search, and Bayesian estimation.

15. A computer-implemented method, comprising:
obtaining, from a plurality of mobile devices, each mobile device associated with one or more of a plurality of patients in a patient population, a plurality of game results, wherein:
each game result in the plurality of game results comprises game boards, moves provided by a patient as input to the mobile device providing the game result, and a difficulty for each of the game boards;
the patient population comprises at least one characteristic in common between each patient in the patient population;
determining, based on the plurality of game results, the moves provided by the patient for each of the plurality of game results, and the difficulty for each of the game boards in the game results, sorted game results;
iteratively generating representative parameters for each of the plurality of game results; and
determining, for each patient and based on the iteratively generated representative parameters associated with the patient, a spatial working memory score for the patient.

16. The computer-implemented method of claim 15, wherein the patient population comprises a subset of the population that has been administered a treatment, and wherein the method further comprises:
obtaining a historical memory score for each patient in the subset of the patient population; and
determining, based on a comparison of the historical memory score and the spatial working memory score for each patient in the subset of the patient population, a treatment effectiveness for the administered treatment.

17. The computer-implemented method of claim 16, further comprising:
obtaining, from a second plurality of mobile devices associated with a second subset of the patient population, a second plurality of game results, wherein:
each game result in the second plurality of game results comprises game boards and moves provided by a patient associated with the mobile device providing the game result;
the second subset comprises patients administered a second treatment and at least one second characteristic in common between each patient in the second patient population;
determining, for each patient in the second subset, a second spatial working memory score for each patient in the second subset; and
determining the treatment effectiveness for the administered treatment is further based on the second spatial working memory score for each patient in the second subset of the patient population.

18. The computer-implemented method of claim 17, wherein:
the second treatment comprises a placebo; and
the at least one second characteristic of the second patient population is the same as at least one characteristic of the patient population.

19. The computer-implemented method of claim 15, wherein the administered treatment comprises a therapeutically effective dose of a drug selected from the group consisting of Arbaclofen, Balovaptan, a GABA-Aa5 PAM, a GABA-A1 modulator, a mGlu4/7 PAM, a Dopamine 2 receptor antagonist, in particular Risperidone, mu-opioid receptor antagonist, in particular naloxone, and/or NMDA glutamate receptor antagonist, in particular memantine, and pharmaceutically acceptable salts thereof.

20. The computer-implemented method of claim 15, wherein the representative parameters are iteratively generated using a procedure selected from the group consisting of Markov Chain Monte Carlo simulations, grid search, and Bayesian estimation.
